(19) United States
(12) Patent Application Publication
(10) Pub. No.: US 2015/0368197 A1
(43) Pub. Date: Dec. 24, 2015

(54) COMPOSITIONS AND METHODS FOR TREATMENT OF INFLAMMATORY DISEASES OF THE LUNG

(71) Applicant: RADIKAL THERAPEUTICS INS., West Tisbury, MA (US)

(72) Inventors: Andrew Lurie Salzman, West Tisbury, MA (US); Prakash Jagtap, Andover, MA (US); Garry John Southan, c, MA (US)

(21) Appl. No.: 14/410,024

(22) PCT Filed: Jun. 20, 2013
(86) PCT No.: PCT/IB2013/055067

§ 371 Date: Dec. 19, 2014

Related U.S. Application Data
(60) Provisional application No. 61/662,611, filed on Jun. 21, 2012.

(51) Int. Cl. C07D 211/60; C07D 209/42; C07D 207/09; C07D 209/52; C07D 203/08; C07D 205/04

(52) U.S. Cl. CPC C07D 211/60; C07D 203/08; C07D 205/04; C07D 207/09; C07D 209/52; C07D 209/42

(57) ABSTRACT

Pharmaceutical compositions and methods are for treatment of an inflammatory disease of the lung caused by inhalation of a toxic agent or an irritant. In one example, chlorine inhalational lung injury can be treated using compounds useful in such compositions.

### TECHNICAL FIELD

The present invention relates to pharmaceutical compositions and methods for treatment of inflammatory diseases of the lung caused by inhalation of toxic agents such as chlorine (Cl₂), or irritants.

### BACKGROUND ART

Acute exposure of animals to high levels of Cl₂ gas induces a disease characterized by severe oxidative stress of the peripheral airways (Yadav et al., 2010) as Cl₂ and its breakdown product, hypochlorous acid, react directly with biological molecules in the lung epithelial lining fluid (Squadrito et al., 2010). This results in injury to the lung and impairs its function, and can lead to incapacitation and death. In response to oxidative stress, the lung undergoes mucosal apoptosis and necrosis, alveolar edema, polymorphonuclear neutrophil (PMN) infiltration (Hoyle, 2010; Tian et al., 2008), disruption of airway epithelial tight junctions (Guo et al., 1996), pulmonary arterial hypertension (PAH) (Batchinsky et al., 2006), and pulmonary shunt and hypoxemia (Yadav et al., 2010).

U.S. Pat. Nos. 6,448,267, 6,455,542 and 6,759,430, herewith incorporated by reference in their entirety as if fully described herein, disclose, inter alia, piperidine, pyrrolidine and azepane derivatives comprising a nitric oxide (NO) donor and a superoxide scavenger, capable of acting as sources of NO and as reactive oxygen species (ROS) degradation catalysts, their preparation, and their use in the treatment of various conditions associated with oxidative stress or endothelial dysfunction such as diabetes mellitus and cardiovascular diseases.

International Publication Nos. WO 2011/092690, WO 2012/093383 and WO 2013/005216 disclose methods and pharmaceutical compositions for treatment of pulmonary arterial hypertension, sepsis and conditions associated therewith, and renal ischemia-reperfusion injury, respectively, using the piperidine, pyrrolidine, and azepane derivatives disclosed in U.S. Pat. Nos. 6,448,267, 6,455,542 and 6,759,430.

1-(2-acetamido-3-mercaptopropanoyl)-N-(1-amino-3-mercapto-1-oxopropan-2-yl)pyrrolidine-2-carboxamide, 2-(1-(2-amino-3-mercaptopropanoyl) pyrrolidine-2-carboxamido)-3-mercaptopropanoic acid, and analogues thereof are thioredoxin (TRX) mimetics, thiol-rich tripeptide containing cysteine-proline-cysteine (Cys-Pro-Cys) or analogues, which are closely analogous to the native TRX motif. TRX is a multifunctional redox-active protein that scavenges reactive oxygen species (ROS) by itself or together with TRX-dependent peroxiredoxin, and is a critical element in the defense against redox stress. TRX also has chemotaxis-modulating functions and suppresses PMN infiltration into sites of inflammation (Hoshino et al., 2003). The redox stress of acute lung injury (ALI) is initially countered by endogenous reductants, especially TRX, but such thiol-rich reductant defenses are readily overwhelmed by massive oxidant insults. The subsequent depletion of TRX increases susceptibility to ALI, as noted in models of hyperoxic lung injury (Tipple et al., 2007). 1-(2-acetamido-3-mercaptopropanoyl)-N-(1-amino-3-mercapto-1-oxopropan-2-yl)pyrrolidine-2-carboxamide has been shown to be efficacious in a murine model of asthma induced by sensitization and challenge with ovalbumin, but was not recognized as having efficacy in halogen induced lung inhalation injury.

### SUMMARY OF INVENTION

It has been found in accordance with the present invention that the aforesaid 2-(1-(2-amino-3-mercaptopropanoyl)pyrrolidine-2-carboxamido)-3-mercaptopropanoic acid, representing the amino-acid sequence Cys-Pro-Cys, and 1-(2-acetamido-3-mercapto propanoyl)-N-(1-amino-3-mercapto-1-oxopropan-2-yl)pyrrolidine-2-carboxamide, representing said amino-acid sequence wherein the terminal amino group is acylated and the terminal carboxyl group is amidated; as well as certain 1-pyrrolidinyloxy derivatives, more particularly 3-nitratomethyl-2,2,5,5-tetramethylpyrrolidinyloxy, are effective as a rescue therapy in a murine model of acute Cl₂ inhalational lung injury (CILI), more particularly, ameliorate the effects of exposure to Cl₂ as seen by observing lung morphology, biochemical markers and mortality rate.

In one aspect, the present invention thus relates to a method for treatment of an inflammatory disease of the lung caused by inhalation of a toxic agent or an irritant, in an individual in need thereof, said method comprising administering to said individual a therapeutically effective amount of a compound of the general formula II:

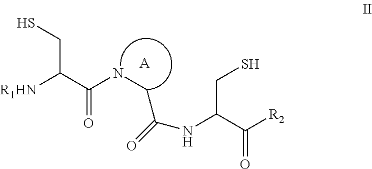

or an enantiomer, diastereomer, racemate, or a pharmaceutically acceptable salt or solvate thereof,

wherein

R₁ is H, —CO(C₁-C₈)alkyl, —COO(C₁-C₈)alkyl or —CONH(C₁-C₈)alkyl;

R₂ is OH, or N(R₃R₄);

R₃ and R₄ each independently is H, (C₁-C₈)alkyl, (C₃-C₁₀)cycloalkyl, 4-12-membered heterocyclyl, or (C₆-C₁₄)aryl;

A is a 3-6 membered ring optionally containing one or more additional heteroatoms selected from sulfur, oxygen or nitrogen, wherein said nitrogen atom may be substituted by (C₁-C₈)alkyl, and each one of the carbon atoms in said ring may be substituted by oxo, halogen, (C₁-C₈)alkyl, (C₆-C₁₄ aryl, 4-12-membered heterocyclyl, NO₂, N(R₅R₆), —OR₅, —SR₅, —SO₂R₅, or —COR₇, or two adjacent carbon atoms in said ring form a 3-6 membered saturated, partially saturated, or aromatic carbocyclic or heterocyclic ring;

R₅ and R₆ each independently is H, or (C₁-C₈)alkyl; and

R₇ is OH, NH₂, or —O(C₁-C₈)alkyl.

In another aspect, the present invention relates to a method for treatment of an inflammatory disease of the lung caused by inhalation of a toxic agent or an irritant, in an individual in need thereof, said method comprising administering to said individual a therapeutically effective amount of a compound of the general formula I:

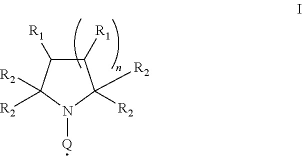

or an enantiomer, diastereomer, racemate, or pharmaceutically acceptable salt or solvate thereof,

wherein

R₁ each independently is selected from H, —OH, —COR₃, —COOR₃, —OCOOR₃, —OCON(R₃)₂, —(C₁-C₁₆)alkylene-COOR₃, —CN, —NO₂, —SH, —SR₃, —(C₁-C₁₆)alkyl, —O—(C₁-C₁₆)alkyl, —N(R₃)₂, —CON(R₃)₂, —SO₂R₃, —S(═O)R₃, or a nitric oxide donor group of the formula —X₁-X₂-X₃, wherein X₁ is absent or selected from —O—, —S— or —NH—; X₂ is absent or is (C₁-C₂₀)alkylene optionally substituted by one or more —ONO₂ groups and optionally further substituted by a moiety of the general formula D:

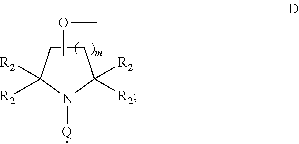

and X₃ is —NO or —ONO₂, provided that at least one R₁ group is a nitric oxide donor group;

R₂ each independently is selected from (C₁-C₁₆)alkyl, (C₂-C₁₆)alkenyl, or (C₂-C₁₆)alkynyl;

R₃ each independently is selected from H, (C₁-C₈)alkyl, (C₃-C₁₀)cycloalkyl, 4-12-membered heterocyclyl, or (C₆-C₁₄)aryl, each of which other than H may optionally be substituted with —OH, —COR₄, —COOR₄, —OCOOR₄, —OCON(R₄)₂, —(C₁-C₈)alkylene-COOR₄, —CN, —NO₂, —SH, —SR₄, —(C₁-C₈)alkyl, —O—(C₁-C₈)alkyl, —N(R₄)₂, —CON(R₄)₂, —SO₂R₄, or —S(═O)R₄;

R₄ each independently is selected from H, (C₁-C₈)alkyl, (C₃-C₁₀)cycloalkyl, 4-12-membered heterocyclyl, or (C₆-C₁₄)aryl; and

n and m each independently is an integer of 1 to 3.

In a further aspect, the present invention provides a pharmaceutical composition for treatment of an inflammatory disease of the lung caused by inhalation of a toxic agent or an irritant, said composition comprising a pharmaceutically acceptable carrier and a compound of the general formula I or II as defined above, or an enantiomer, diastereomer, racemate, or pharmaceutically acceptable salt or solvate thereof.

In yet a further aspect, the present invention provides a compound of the general formula I or II as defined above, or an enantiomer, diastereomer, racemate, or pharmaceutically acceptable salt or solvate thereof, for use in treatment of an inflammatory disease of the lung caused by inhalation of a toxic agent or an irritant.

In still a further aspect, the present invention relates to use of a compound of the general formula I or II as defined above, or an enantiomer, diastereomer, racemate, or pharmaceutically acceptable salt or solvate thereof, for the preparation of a pharmaceutical composition for treatment of an inflammatory disease of the lung caused by inhalation of a toxic agent or an irritant.

In particular embodiments, the inflammatory disease of the lung treated by the methods and compositions of the present invention is CILI, caused by inhalation of Cl₂.

In yet another aspect, the present invention provides a compound of the general formula II as defined above, or an enantiomer, diastereomer, racemate, or pharmaceutically acceptable salt or solvate thereof, but excluding the compounds wherein R₁ is H or —COCH₃, R₂ is OH or NH₂, and A is pyrrolidin-1,2-diyl.

In still another aspect, the present invention provides a pharmaceutical composition comprising a compound of the general formula II as defined above, or an enantiomer, diastereomer, racemate, or pharmaceutically acceptable salt or solvate thereof, but excluding the compounds wherein R₁ is H or —COCH₃, R₂ is OH or NH₂, and A is pyrrolidin-1,2-diyl, and a pharmaceutically acceptable carrier.

### BRIEF DESCRIPTION OF DRAWINGS

FIG. 1 shows that R-100, when administered 2 and 6 hours post a 30 minute exposure to Cl₂-containing air, dose-dependently attenuated CILI in mice 24 hours post exposure as exemplified by the improved histology scores. A (Cl₂+HPCD); B (Cl₂+R-100, 4 mg/kg/dose); C (Cl₂+R-100, 12 mg/kg/dose); D (Cl₂+R-100, 40 mg/kg/dose); and E (Cl₂+R-100, 80 mg/kg/dose).

FIG. 2 shows that R-907, when administered 2 and 6 hours post a 30 minute exposure to Cl₂-containing air, does-dependently attenuated CILI in mice 24 hours post exposure as exemplified by the improved histology scores. A (Cl₂+vehicle); B (Cl₂+R-907, 3 mg/kg/dose); C (Cl₂+R-907, 10 mg/kg/dose); D (Cl₂+R-907, 30 mg/kg/dose); and E (Cl₂+R-907, 80 mg/kg/dose).

FIGS. 3A-3B show that R-901 therapy as described in Example 3 reduced the elevation in pulmonary MPO level (3A) and histological damage (3B) by 50% (p<0.0001) and 20% (n.s.), respectively, relative to placebo.

FIG. 4 shows that both R-100 and R-907, when administered 2 and 6 hours post a 60 minute exposure to Cl₂-containing air at concentrations of 1, 3, 10 or 30 mg/kg/dose (R-100) and 3, 10, 30 or 80 mg/kg/dose (R-907), attenuate CILI in mice 24 hours post exposure as exemplified by the improved histology scores.

FIGS. 5A-5B show the beneficial effects of R-100 (5A) and R-907 (5B) on the survival and weight loss of the animals after a 60 minute exposure to 400 ppm Cl₂-containing air.

FIG. 6 shows the MS data of compound 33 ((ES⁺): m/z 362.20 (M+1)).

### DETAILED DESCRIPTION OF THE INVENTION

In one aspect, the present invention relates to a method for treatment of an inflammatory disease of the lung caused by inhalation of a toxic agent or an irritant by administration of a peptide or a peptidomimetic of the general formula II as defined above, representing the amino-acid sequence Cys-Pro-Cys or a derivative thereof in which the pyrrolidin-1,2-diyl of the proline residue is replaced by a 3-6 heterocyclil represented by the group A in the general formula II.

In another aspect, the present invention relates to a method for treatment of an inflammatory disease of the lung caused by inhalation of a toxic agent or an irritant by administration of piperidine, pyrrolidine, or azepane derivatives of the general formula I as defined above, comprising one to four NO donor groups and a reactive oxygen species (ROS) degradation catalyst, i.e., a superoxide scavenger.

The term “alkyl” as used herein typically means a straight or branched saturated hydrocarbon radical having 1-16 carbon atoms and includes, e.g., methyl, ethyl, n-propyl, isopropyl, n-butyl, sec-butyl, isobutyl, tert-butyl, n-pentyl, 2,2-dimethylpropyl, n-hexyl, n-heptyl, n-octyl, n-nonyl, n-decyl, n-undecyl, n-dodecyl, n-tridecyl, n-tetradecyl, n-pentadecyl, n-hexadecyl, and the like. Preferred are (C₁-C₆)alkyl groups, more preferably (C₁-C₄)alkyl groups, most preferably methyl and ethyl. The terms “alkenyl” and “alkynyl” typically mean straight and branched hydrocarbon radicals having 2-16 carbon atoms and 1 double or triple bond, respectively, and include ethenyl, propenyl, 3-buten-1-yl, 2-ethenylbutyl, 3-octen-1-yl, 3-nonenyl, 3-decenyl, and the like, and propynyl, 2-butyn-1-yl, 3-pentyn-1-yl, 3-hexynyl, 3-octynyl, 4-decynyl, and the like. C₂-C₆ alkenyl and alkynyl radicals are preferred, more preferably C₂-C₄ alkenyl and alkynyl.

The term “alkylene” typically means a divalent straight or branched hydrocarbon radical having 1-20 carbon atoms and includes, e.g., methylene, ethylene, propylene, butylene, 2-methylpropylene, pentylene, 2-methylbutylene, hexylene, 2-methylpentylene, 3-methylpentylene, 2,3-dimethylbutylene, heptylene, octylene and the like. Preferred are (C₁-C₈)alkylene, more preferably (C₁-C₄)alkylene, most preferably (C₁-C₂)alkylene.

The term “cycloalkyl” as used herein means a cyclic or bicyclic hydrocarbyl group having 3-12 carbon atoms such as cyclopropyl, cyclobutyl, cyclopentyl, cyclohexyl, cycloheptyl, cyclooctyl, adamantyl, bicyclo[3.2.1]octyl, bicyclo[2.2.1]heptyl, and the like. Preferred are (C₅-C₁₀)cycloalkyls, more preferably (C₅-C₇)cycloalkyls.

The term “aryl” denotes an aromatic carbocyclic group having 6-14 carbon atoms consisting of a single ring or multiple rings either condensed or linked by a covalent bond such as, but not limited to, phenyl, naphthyl, phenanthryl, and biphenyl.

The term “heterocyclic ring” denotes a mono- or poly-cyclic non-aromatic ring of 4-12 atoms containing at least one carbon atom and one to three heteroatoms selected from sulfur, oxygen or nitrogen, which may be saturated or unsaturated, i.e., containing at least one unsaturated bond. Preferred are 5- or 6-membered heterocyclic rings. The term “heterocyclyl” as used herein refers to any univalent radical derived from a heterocyclic ring as defined herein by removal of hydrogen from any ring atom. Examples of such radicals include, without limitation, piperidino, 4-morpholinyl, or pyrrolidinyl.

The term “nitric oxide donor group” as defined herein refers to any group of the formula —X₁-X₂-X₃, wherein X₁ may be absent or is selected from —O—, —S— or —NH—; X₂ may be absent or is (C₁-C₂₀)alkylene optionally substituted by one or more —ONO₂ groups and optionally further substituted by a moiety of the general formula D as defined above; and X₃ is —NO or —ONO₂. Preferred nitric oxide donor groups are those in which X₁ is absent or is —O—; X₂ is absent or is —(C₁-C₆)alkylene, preferably —(C₁-C₄)alkylene, more preferably methylene; and X₃ is —NO or —ONO₂, preferably —ONO₂, and said alkylene is optionally substituted as defined hereinabove. According to the method of the present invention, the compound of the general formula I may comprise one nitric oxide donor group or more than one identical or different nitric oxide donor groups.

### Methods for Treatment Using Compounds of the General Formula I

In certain embodiments, the compound used according to the method of the present invention is a compound of the general formula I, wherein R₁ each independently is selected from H, —COOR₃, —CON(R₃)₂, or a nitric oxide donor group; and R₃ is H.

In certain embodiments, the compound used according to the method of the present invention is a compound of the general formula I, wherein R₂ each independently is (C₁-C₈)alkyl, preferably (C₁-C₄)alkyl, more preferably (C₁-C₂)alkyl, most preferably methyl. Preferred embodiments are those in which all the R₂ groups in the formula I are identical.

In certain embodiments, the compound used according to the method of the present invention is a compound of the general formula I, wherein in said nitric oxide donor group, X₁ is absent or —O—; X₂ is absent or (C₁-C₂₀)alkylene, preferably —(C₁-C₆)alkylene, more preferably —(C₁-C₄)alkylene, most preferably methylene; X₃ is —NO or —ONO₂, preferably —ONO₂; and said alkylene is optionally substituted by one or more —ONO₂ groups and optionally further substituted by a moiety of the general formula D as defined above.

In certain embodiments, the compound used according to the method of the present invention is a compound of the general formula I, wherein n is 1, 2 or 3, preferably 1 or 2.

In certain embodiments, the compound used according to the method of the present invention has the general formula I, wherein n is 1, i.e., a 1-pyrrolidinyloxy derivative of the formula Ia (see Table 1). In particular embodiments, the compound used according to this method has the general formula Ia, wherein either the carbon atom at position 3 of the pyrrolidine ring or the carbon atom at position 4 of the pyrrolidine ring, or both, are each linked to a nitric oxide donor group.

In other certain embodiments, the compound used according to the method of the present invention has the general formula I, wherein n is 2, i.e., a 1-piperidinyloxy derivative of the formula Ib (see Table 1). In particular embodiments, the compound used according to this method has the general formula Ib, wherein one, two or three of the carbon atoms at positions 3 to 5 of the piperidine ring are each linked to a nitric oxide donor group. In more particular embodiments, (i) the carbon atom at position 3 of the piperidine ring and optionally one or more of the carbon atoms at positions 4 or 5 of the piperidine ring are each linked to a nitric oxide donor group; (ii) the carbon atom at position 4 of the piperidine ring and optionally one or more of the carbon atoms at positions 3 or 5 of the piperidine ring are each linked to a nitric oxide donor group; or (iii) the carbon atom at position 5 of the piperidine ring and optionally one or more of the carbon atoms at positions 3 or 4 of the piperidine ring are each linked to a nitric oxide donor group.

In further certain embodiments, the compound used according to the method of the present invention has the general formula I, wherein n is 3, i.e., a 1-azepanyloxy derivative of the formula Ic (see Table 1). In particular embodiments, the compound used according to this method has the general formula Ic, wherein one, two, three or four of the carbon atoms at positions 3 to 6 of the azepane ring are each linked to a nitric oxide donor group. In more particular embodiments, (i) the carbon atom at position 3 of the azepane ring and optionally one or more of the carbon atoms at positions 4 to 6 of the azepane ring are each linked to a nitric oxide donor group; (ii) the carbon atom at position 4 of the azepane ring and optionally one or more of the carbon atoms at positions 3, 5 or 6 of the azepane ring are each linked to a nitric oxide donor group; (iii) the carbon atom at position 5 of the azepane ring and optionally one or more of the carbon atoms at positions 3, 4 or 6 of the azepane ring are each linked to a nitric oxide donor group; or (iv) the carbon atom at position 6 of the azepane ring and optionally one or more of the carbon atoms at positions 3 to 5 of the azepane ring are each linked to a nitric oxide donor group.

In particular embodiments, the compound used according to the method of the invention is a 1-pyrrolidinyloxy derivative of the formula Ia, 1-piperidinyloxy derivative of the formula Ib, or 1-azepanyloxy derivative of the formula Ic, and each one of the nitric oxide donor groups in said compound independently is of the formula —(C₁-C₆)alkylene-ONO₂, preferably —(C₁-C₄)alkylene-ONO₂, more preferably —CH₂—ONO₂, or —O—(C₁-C₆)alkylene-ONO₂, wherein said alkylene is optionally substituted by one or more —ONO₂ groups, or is —ONO₂.

TABLE 1
Structures Ia, Ib and Ic, indicating 1-pyrrolidinyloxy,
1-piperidinyloxy and 1-azepanyloxy derivatives, respectively

| | |
|---|---|
| 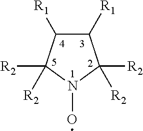 | Ia |
| 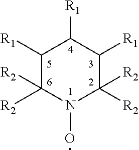 | Ib |
| 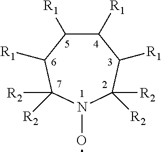 | Ic |

Specific compounds of the general formulas Ia, Ib and Ic described herein, in which each one of the R₁ groups independently is either H or the nitric oxide donor group —CH₂—ONO₂ or —ONO₂, are herein identified compounds 1a/b-15a/b in bold (compound 1a is also identified R-100), and their full chemical structures are depicted in Table 2. Other specific compounds of the general formulas Ia and Ib described herein, in which one R₁ group is the nitric oxide donor group —CH₂—ONO₂ or —ONO₂, and another R₁ group is not H, are herein identified compounds 16a/b-17a/b in bold, and their full chemical structures are depicted in Table 3. A further specific compound of the general formula Ib described herein, in which one R₁ group is the nitric oxide donor group —O—CH₂—CH(ONO₂)CH₂—ONO₂, and the other R₁ groups are H, is herein identified compound 18 in bold, and its full chemical structure is depicted in Table 3.

In specific embodiments, the compound used according to the method of the invention is the compound of formula Ia, i.e., a compound of the general formula I in which n is 1, wherein R₂ each is methyl; and (i) the R₁ group linked to the carbon atom at position 3 of the pyrrolidine ring is the nitric oxide donor group —CH₂—ONO₂ or ONO₂; and the R₁ group linked to the carbon atom at position 4 of the pyrrolidine ring is H, i.e., 3-nitratomethyl-2,2,5,5-tetramethylpyrrolidinyloxy (compound 1a; R-100) or 3-nitrato-2,2,5,5-tetramethylpyrrolidinyloxy (compound 1b), respectively; or (ii) each one of the R₁ groups linked to the carbon atoms at positions 3 and 4 of the pyrrolidine ring is the nitric oxide donor group —CH₂—ONO₂ or ONO₂, i.e., 3,4-dinitratomethyl-2,2,5,5-tetramethyl pyrrolidinyloxy (compound 2a) or 3,4-dinitrato-2,2,5,5-tetramethylpyrrolidinyloxy (compound 2b), respectively.

In other specific embodiments, the compound used according to the method of the invention is the compound of formula Ib, i.e., a compound of the general formula I wherein n is 2, wherein R₂ each is methyl; and (i) the R₁ group linked to the carbon atom at position 3 of the piperidine ring is the nitric oxide donor group —CH₂—ONO₂ or ONO₂; and each one of the R₁ groups linked to the carbon atoms at positions 4 and 5 of the piperidine ring is H, i.e., 3-nitratomethyl-2,2,6,6-tetramethylpiperidinyloxy(3-nitratomethyl-TEMPO; compound 3a) or 3-nitrato-2,2,6,6-tetramethylpiperidinyloxy(3-nitrato-TEMPO; compound 3b), respectively; (ii) the R₁ group linked to the carbon atom at position 4 of the piperidine ring is the nitric oxide donor group —CH₂—ONO₂ or ONO₂; and each one of the R₁ groups linked to the carbon atoms at positions 3 and 5 of the piperidine ring is H, i.e., 4-nitratomethyl-2,2,6,6-tetramethylpiperidinyloxy(4-nitratomethyl-TEMPO; compound 4a) or 4-nitrato-2,2,6,6-tetramethylpiperidinyloxy(3-nitrato-TEMPO; compound 4b), respectively; (iii) each one of the R₁ groups linked to the carbon atoms at positions 3 and 4 of the piperidine ring is the nitric oxide donor group —CH₂—ONO₂ or ONO₂; and the R₁ group linked to the carbon atom at position 5 of the piperidine ring is H, i.e., 3,4-dinitratomethyl-2,2,6,6-tetramethyl piperidinyloxy(3,4-dinitratomethyl-TEMPO; compound 5a) or 3,4-dinitrato-2,2,6,6-tetramethylpiperidinyloxy(3,4-dinitrato-TEMPO; compound 5b), respectively; (iv) each one of the R₁ groups linked to the carbon atoms at positions 3 and 5 of the piperidine ring is the nitric oxide donor group —CH₂—ONO₂ or ONO₂; and the R₁ group linked to the carbon atom at position 4 of the piperidine ring is H, i.e., 3,5-dinitratomethyl-2,2,6,6-tetramethylpiperidinyloxy(3,5-dinitratomethyl-TEMPO; compound 6a) or 3,5-dinitrato-2,2,6,6-tetramethylpiperidinyloxy(3,5-dinitrato-TEMPO; compound 6b), respectively; or (v) each one of the R₁ groups linked to the carbon atoms at positions 3 to 5 of the piperidine ring is the nitric oxide donor group —CH₂—ONO₂ or ONO₂, i.e., 3,4,5-trinitratomethyl-2,2,6,6-tetramethylpiperidinyloxy(3,4,5-trinitratomethyl-TEMPO; compound 7a) or 3,4,5-trinitrato-2,2,6,6-tetramethylpiperidinyloxy(3,4,5-trinitrato-TEMPO; compound 7b), respectively.

In further specific embodiments, the compound used according to the method of the invention is the compound of formula Ic, i.e., a compound of the general formula I wherein n is 3, wherein R₂ each is methyl; and (i) the R₁ group linked to the carbon atom at position 3 of the azepane ring is the nitric oxide donor group —CH₂—ONO₂ or ONO₂; and each one of the R₁ groups linked to the carbon atoms at positions 4 to 6 of the azepane ring is H, i.e., 3-nitratomethyl-2,2,7,7-tetramethylazepanyloxy (compound 8a) or 3-nitrato-2,2,7,7-tetramethylazepanyloxy (compound 8b), respectively; (ii) the R₁ group linked to the carbon atom at position 4 of the azepane ring is the nitric oxide donor group —CH₂—ONO₂ or ONO₂; and each one of the R₁ groups linked to the carbon atoms at position 3, 5 and 6 of the azepane ring is H, i.e., 4-nitratomethyl-2,2,7,7-tetramethylazepanyloxy (compound 9a) or 4-nitrato-2,2,7,7-tetramethylazepanyloxy (compound 9b), respectively; (iii) each one of the R₁ groups linked to the carbon atoms at positions 3 and 4 of the azepane ring is the nitric oxide donor group —CH₂—ONO₂ or ONO₂; and each one of the R₁ groups linked to the carbon atoms at positions 5 and 6 of the azepane ring is H, i.e., 3,4-dinitratomethyl-2,2,7,7-tetramethylazepanyloxy (compound 10a) or 3,4-dinitrato-2,2,7,7-tetramethylazepanyloxy (compound 10b), respectively; (iv) each one of the R₁ groups linked to the carbon atoms at positions 3 and 5 of the azepane ring is the nitric oxide donor group —CH₂—ONO₂ or ONO₂; and each one of the R₁ groups linked to the carbon atoms at positions 4 and 6 of the azepane ring is H, i.e., 3,5-dinitratomethyl-2,2,7,7-tetramethyl azepanyloxy (compound 11a) or 3,5-dinitrato-2,2,7,7-tetramethylazepanyloxy (compound 11b), respectively; (v) each one of the R₁ groups linked to the carbon atoms at positions 3 and 6 of the azepane ring is the nitric oxide donor group —CH₂—ONO₂ or ONO₂; and each one of the R₁ groups linked to the carbon atoms at positions 4 and 5 of the azepane ring is H, i.e., 3,6-dinitratomethyl-2,2,7,7-tetramethylazepanyloxy (compound 12a) or 3,6-dinitrato-2,2,7,7-tetramethylazepanyloxy (compound 12b), respectively; (vi) each one of the R₁ groups linked to the carbon atoms at positions 3 to 5 of the azepane ring is the nitric oxide donor group —CH₂—ONO₂ or ONO₂; and the R₁ group linked to the carbon atom at position 6 of the azepane ring is H, i.e., 3,4,5-trinitratomethyl-2,2,7,7-tetramethyl azepanyloxy (compound 13a) or 3,4,5-trinitrato-2,2,7,7-tetramethylazepanyloxy (compound 13b), respectively; (vii) each of the R₁ groups linked to the carbon atoms at positions 3, 4 and 6 of the azepane ring is the nitric oxide donor group —CH₂—ONO₂ or ONO₂; and the R₁ group linked to the carbon atom at position 5 of the azepane ring is H, i.e., 3,4,6-trinitratomethyl-2,2,7,7-tetramethylazepanyloxy (compound 14a) or 3,4,6-trinitrato-2,2,7,7-tetramethyl azepanyloxy (compound 14b), respectively); or (viii) each of the R₁ groups linked to the carbon atoms at positions 3 to 6 of the azepane ring is the nitric oxide donor group —CH₂—ONO₂ or ONO₂, i.e., 3,4,5,6-tetranitratomethyl-2,2,7,7-tetramethylazepanyloxy (compound 15a) or 3,4,5,6-tetranitrato-2,2,7,7-tetramethyl azepanyloxy (compound 15b), respectively.

In still other specific embodiments, the compound used according to the method of the invention is the compound of formula Ia, wherein R₂ each is methyl; the R₁ group linked to the carbon atom at position 3 of the pyrrolidine ring is the nitric oxide donor group —CH₂—ONO₂ or —ONO₂; and the R₁ group linked to the carbon atom at position 4 of the pyrrolidine ring is —CONH₂, i.e., 3-nitratomethyl-4-carbamoyl-2,2,5,5-tetramethyl pyrrolidinyloxy (compound 16a) or 3-nitrato-4-carbamoyl-2,2,5,5-tetramethyl pyrrolidinyloxy (compound 16b), respectively.

In yet other specific embodiments, the compound used according to the method of the invention is the compound of formula Ib, wherein R₂ each is methyl; the R₁ group linked to the carbon atom at position 3 of the piperidine ring is the nitric oxide donor group —CH₂—ONO₂ or —ONO₂; the R₁ group linked to the carbon atom at position 4 of the piperidine ring is —COOH; and the R₁ group linked to the carbon atoms at position 5 of the piperidine ring is H, i.e., 3-nitratomethyl-4-carboxy-2,2,6,6-tetramethylpiperidinyloxy(3-nitratomethyl-4-carboxy-TEMPO; compound 17a) or 3-nitrato-4-carboxy-2,2,6,6-tetramethylpiperidinyloxy(3-nitrato-4-carboxy-TEMPO; compound 17b), respectively.

In still a further specific embodiment, the compound used according to the method of the invention is the compound of formula Ib, wherein R₂ each is methyl; the R₁ group linked to the carbon atom at position 4 of the piperidine ring is the nitric oxide donor group —O—CH₂—CH(ONO₂)CH₂—ONO₂; and each one of the R₁ groups linked to the carbon atom at position 3 and 5 of the piperidine ring is H, i.e., 4-(2,3-dinitratopropyloxy)-2,2,6,6-tetramethylpiperidinyloxy(4-(2,3-dinitratopropyloxy)-TEMPO; compound 18).

TABLE 2
Compounds of the general formulas Ia, Ib and Ic, identified 1a-15a*

| | |
|---|---|
| 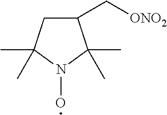 | 1a |
| 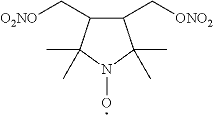 | 2a |
| 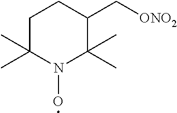 | 3a |
| 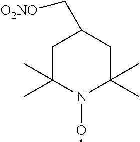 | 4a |
| 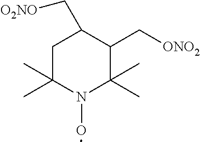 | 5a |
| 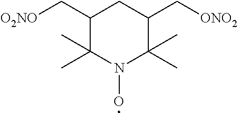 | 6a |
| 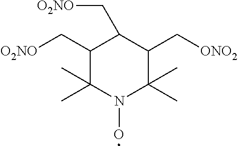 | 7a |
| 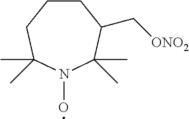 | 8a |
| 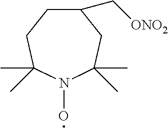 | 9a |
| 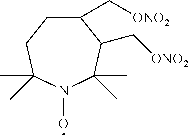 | 10a |
| 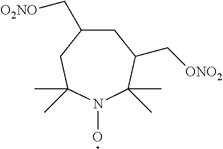 | 11a |
| 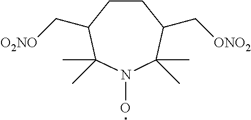 | 12a |
| 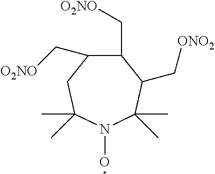 | 13a |
| 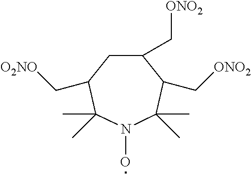 | 14a |
| 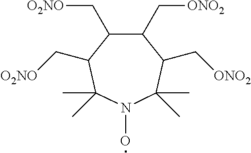 | 15a |

*The compounds corresponding to 1a-15a, in which each one of the —CH₂—ONO₂ groups is replaced by the —ONO₂ group, are identified compounds 1b-15b

TABLE 3
Compounds of the general formulas Ia
and Ib, identified 16a-17a* and 18

| 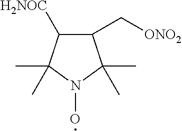 | 16a |
|---|---|
| 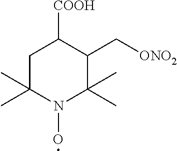 | 17a |
| 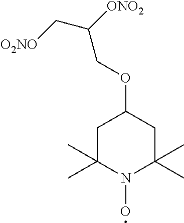 | 18 |

*The compounds corresponding to 16a and 17a, in which each one of the —CH₂—ONO₂ groups is replaced by the —ONO₂ group, are identified compounds 16b and 17b

In other particular embodiments, the compound used according to the method of the present invention is a 1-pyrrolidinyloxy derivative of the formula Ia, 1-piperidinyloxy derivative of the formula Ib, or 1-azepanyloxy derivative of the formula Ic; wherein at least one of the nitric oxide donor groups in said compound is of the formula —O—(C₁-C₆)alkylene-ONO₂; and said alkylene is substituted by a moiety of the general formula D as defined above, and is optionally further substituted by one or more —ONO₂ groups. The general formula D, in which oxygen atom is linked to the carbon atom at position 3 or 4 of the ring, represents a 3-hydroxy-pyrrolidinoxy, 3- or 4-hydroxy-piperidinyloxy, or 3- or 4-hydroxy-azepanyloxy derivative. Conceptually, the compound used in this case is thus a dimer- or higher multimer-like compound, in which two or more identical or different entities, each independently being selected from 1-pyrrolidinyloxy, 1-piperidinyloxy or 1-azepanyloxy derivatives, are linked via alkylene bridges substituted by one or more —ONO₂ groups, wherein each alkylene bridge links two entities only.

Preferred dimer- or higher multimer-like compounds to be used according to the method of the invention are those in which (i) a 1-pyrrolidinyloxy derivative of the general formula Ia is linked via one or two nitric oxide donor groups thereof to one or two identical or different moieties of a 3-hydroxy-pyrrolidinoxy, i.e., one or two moieties of the general formula D in which m is 1; (ii) a 1-piperidinyloxy derivative of the general formula Ib is linked via one, two or three nitric oxide donor groups thereof to one, two or three identical or different moieties of a 3-, or 4-hydroxy-piperidinyloxy, i.e., one to three moieties of the general formula D in which m is 2; or (iii) a 1-azepanyloxy derivative of the general formula Ic is linked via one, two, three or four nitric oxide donor groups thereof to one, two, three or four identical or different moieties of a 3-, or 4-hydroxy-azepanyloxy, i.e., one to four moieties of the general formula D in which m is 3.

Specific compounds of the general formula Ib described herein, having a dimer-like structure, are herein identified compounds 19-20 in bold, and their full chemical structures are depicted in Table 4.

In specific embodiments, the compound used according to the method of the invention is the dimer-like compound of formula Ib, wherein each one of R₁ linked to the carbon atoms at positions 3 and 5 of the piperidine ring is H; and (i) R₁ linked to the carbon atom at position 4 of the piperidine ring is the nitric oxide donor group —O—CH₂—CH₂—CH(CH₃)—ONO₂, wherein the 1,3 butane diyl is substituted at position 2 with —ONO₂ group and at position 4 with a moiety of the general formula D, wherein m is 2, and the oxygen atom is linked to the carbon atom at position 4 of the piperidine ring in the formula D; and R₂ each is methyl, i.e., 1,4-di-(4-oxo-TEMPO)-2,3-dinitratobutane (compound 19); or (ii) R₁ linked to the carbon atom at position 4 of the piperidine ring is the nitric oxide donor group —O—CH₂—CH(CH₃)—ONO₂, wherein the 1,2 propane diyl is substituted at position 3 with a moiety of the general formula D, wherein m is 2, and the oxygen atom is linked to the carbon atom at position 4 of the piperidine ring in the formula D; and R₂ each is methyl, i.e., 1,3-di-(4-oxo-TEMPO)-2-nitratopropane (compound 20).

TABLE 4
Compounds of the general formula Ib, identified 19-20

| | |
|---|---|
| 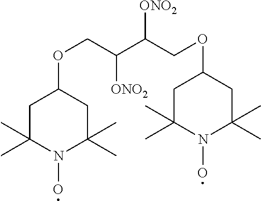 | 19 |
| 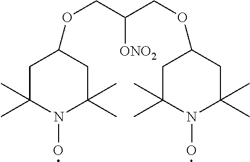 | 20 |

The compounds of the general formula I may be synthesized according to any technology or procedure known in the art, e.g., as described in detail in U.S. Pat. No. 6,448,267, U.S. Pat. No. 6,455,542 and U.S. Pat. No. 6,759,430.

The compounds of the general formula I may have one or more asymmetric centers, and may accordingly exist both as enantiomers, i.e., optical isomers (R, S, or racemate, wherein a certain enantiomer may have an optical purity of 90%, 95%, 99% or more) and as diastereoisomers. Specifically, those chiral centers may be, e.g., in each one of the carbon atoms of the 1-pyrrolidinyloxy derivative, 1-piperidinyloxy derivative; and 1-azepanyloxy derivative of the general formulas Ia, Ib and Ic, respectively. It should be understood that according to the method of the present invention, inflammatory diseases of the lung caused by inhalation of toxic agents or irritants can be treated by administration of all such enantiomers, isomers and mixtures thereof, as well as pharmaceutically acceptable salts and solvates thereof.

Optically active forms of the compounds of the general formula I may be prepared using any method known in the art, e.g., by resolution of the racemic form by recrystallization techniques; by chiral synthesis; by extraction with chiral solvents; or by chromatographic separation using a chiral stationary phase. A non-limiting example of a method for obtaining optically active materials is transport across chiral membranes, i.e., a technique whereby a racemate is placed in contact with a thin membrane barrier, the concentration or pressure differential causes preferential transport across the membrane barrier, and separation occurs as a result of the non-racemic chiral nature of the membrane that allows only one enantiomer of the racemate to pass through. Chiral chromatography, including simulated moving bed chromatography, can also be used. A wide variety of chiral stationary phases are commercially available.

As previously discovered by the present inventors, and disclosed in WO 2013/005216, an aqueous solution of a compound of the general formula I having a concentration several times greater than that with commonly used co-solvents can be obtained by stirring said compound in water with an hydroxyalkyl-cyclodextrin such as hydroxyalkyl-β-cyclodextrin, in particular 2-hydroxyalkyl-β-cyclodextrin (HPCD), in ratios typically between 1:10 and 1:20 w/w, depending on the degree of substitution of the cyclodextrin with the hydroxypropyl side chain. Moreover, an aqueous solution containing substantially higher concentration of said compound with HPCD can be achieved by stirring HPCD in distilled water with said compound; filtering and freeze drying the filtrate; and re-dissolving the resulting freeze dried solid, i.e., the lyophilizate, in a volume of water that is less than that originally used to prepare the solution prior to lyophilization.

### Methods for Treatment Using Compounds of the General Formula II

In certain embodiments, the compound used according to the method of the present invention is a compound of the general formula II, wherein R₁ is H, —CO(C₁-C₄)alkyl, preferably —COCH₃ or —COCH₂CH₃, —COO(C₁-C₄)alkyl, preferably —COOCH₃ or —COOCH₂CH₃, or —CONH(C₁-C₄)alkyl, preferably —CONHCH₃ or —CONHCH₂CH₃.

In certain embodiments, the compound used according to the method of the present invention is a compound of the general formula II, wherein R₂ is —OH, or N(R₃R₄), wherein R₃ and R₄ each independently is H, or (C₁-C₄)alkyl, preferably methyl or ethyl.

In certain embodiments, the compound used according to the method of the present invention is a compound of the general formula II, wherein A is a 3-, 4-, 5-, or 6-membered ring, wherein each one of the carbon atoms in said ring may be substituted by oxo, H, halogen, (C₁-C₄)alkyl, NO₂, N(R₅R₆), —OR₅, —SR₅, —SO₂R₅, or —COR₇, or two adjacent carbon atoms in said ring form a 3-6 membered saturated, partially saturated, or aromatic carbocyclic or heterocyclic ring; R₅ and R₆ each independently is H, or (C₁-C₄)alkyl; and R₇ is OH, NH₂, or —O(C₁-C₄)alkyl. In particular such embodiments, A is a 3-6 membered ring, wherein each one of the carbon atoms in said ring may be substituted by oxo, H, halogen, methyl, ethyl, NO₂, —NH₂, OH, —OCH₃, —OCH₂CH₃, —SH, —SCH₃, —SCH₂CH₃, —SO₂H, —SO₂CH₃, —SO₂CH₂CH₃, —COOH, —COOCH₃, —COOCH₂CH₃, or —CONH₂, or two adjacent carbon atoms in said ring form a 3-6 membered saturated, partially saturated, or aromatic carbocyclic or heterocyclic ring.

In particular embodiments, the compound used according to the method of the present invention is a compound of the general formula II as defined above, wherein R₁ is H, or —CO(C₁-C₄)alkyl; R₂ is —OH, or N(R₃R₄), wherein R₃ and R₄ each independently is H, or (C₁-C₄)alkyl; A is a 3-6 membered ring, wherein each one of the carbon atoms in said ring may be substituted by oxo, H, halogen, (C₁-C₄)alkyl, NO₂, N(R₅R₆), —OR₅, —SR₅, —SO₂R₅, or —COR₇, or two adjacent carbon atoms in said ring form a 3-6 membered saturated, partially saturated, or aromatic carbocyclic or heterocyclic ring; R₅ and R₆ each independently is H, methyl or ethyl; and R₇ is OH, NH₂, methoxy or ethoxy. More particular such embodiments are those wherein R₁ is H, —COCH₃, or —COCH₂CH₃; R₂ is OH or N(R₃R₄), wherein R₃ and R₄ each independently is H, methyl, or ethyl; and A is a 3-6 membered ring, wherein each one of the carbon atoms in said ring may be substituted by oxo, H, halogen, methyl, ethyl, NO₂, —NH₂, OH, —OCH₃, —OCH₂CH₃, —SH, —SCH₃, —SCH₂CH₃, —SO₂H, —SO₂CH₃, —SO₂CH₂CH₃, —COOH, —COOCH₃, —COOCH₂CH₃, or —CONH₂, or two adjacent carbon atoms in said ring form a 3-6 membered saturated, partially saturated, or aromatic carbocyclic or heterocyclic ring. Most particular such embodiments are those wherein R₁ is H or —COCH₃; R₂ is OH or NH₂; and A is azeridin-diyl, azetidin-1,2-diyl, pyrrolidin-1,2-diyl, or piperidin-1,2-diyl, wherein each one of the carbon atoms in said ring may be substituted by halogen, or two adjacent carbon atoms in said ring form cyclopropane, cyclobutane, cyclopentane, cyclohexane, or benzene.

Specific compounds of the general formulas II described herein, in which R₁ is H or —COCH₃, and R₂ is OH or NH₂, are herein identified compounds 21-36 in bold (compounds 25 and 26 are also identified R-907 and R-901, respectively), and their full chemical structures are depicted in Table 5.

In specific embodiments, the compound used according to the method of the invention is the compound of formula II, wherein (i) R₁ is H; R₂ is OH; and A is azeridin-diyl, i.e., 2-(1-(2-amino-3-mercaptopropanoyl)aziridine-2-carboxamido)-3-mercapto propanoic acid (compound 21); (ii) R₁ is —COCH₃; R₂ is NH₂; and A is azeridin-diyl, i.e., 1-(2-acetamido-3-mercaptopropanoyl)-N-(1-amino-3-mercapto-1-oxopropan-2-yl) aziridine-2-carboxamide (compound 22); (iii) R₁ is H; R₂ is OH; and A is azetidin-1,2-diyl, i.e., 2-(1-(2-amino-3-mercaptopropanoyl)azetidine-2-carboxamido)-3-mercaptopropanoic acid (compound 23); (iv) R₁ is —COCH₃; R₂ is NH₂; and A is azetidin-1,2-diyl, i.e., 1-(2-acetamido-3-mercaptopropanoyl)-N-(1-amino-3-mercapto-1-oxopropan-2-yl)azetidine-2-carboxamide (compound 24); (v) R₁ is H; R₂ is OH; and A is pyrolidin-1,2-diyl, i.e., 2-(1-(2-amino-3-mercaptopropanoyl)pyrrolidine-2-carboxamido)-3-mercaptopropanoic acid (compound 25; R-907); (vi) R₁ is —COCH₃; R₂ is NH₂; and A is pyrolidin-1,2-diyl, i.e., 1-(2-acetamido-3-mercaptopropanoyl)-N-(1-amino-3-mercapto-1-oxopropan-2-yl) pyrrolidine-2-carboxamide (compound 26; R-901); (vii) R₁ is H; R₂ is OH; and A is piperidin-1,2-diyl, i.e., 2-(1-(2-amino-3-mercaptopropanoyl)piperidine-2-carboxamido)-3-mercaptopropanoic acid (compound 27); (viii) R₁ is —COCH₃; R₂ is NH₂; and A is piperidin-1,2-diyl, i.e., 1-(2-acetamido-3-mercaptopropanoyl)-N-(1-amino-3-mercapto-1-oxopropan-2-yl)piperidine-2-carboxamide (compound 28); (ix) R₁ is H; R₂ is OH; and A is 4-fluoropyrrolidin-1,2-diyl, i.e., 2-(1-(2-amino-3-mercaptopropanoyl)-4-fluoropyrrolidine-2-carboxamido)-3-mercaptopropanoic acid (compound 29); (x) R₁ is —COCH₃; R₂ is NH₂; and A is 4-fluoropyrrolidin-1,2-diyl, i.e., 1-(2-acetamido-3-mercaptopropanoyl)-N-(1-amino-3-mercapto-1-oxopropan-2-yl)-4-fluoropyrrolidine-2-carboxamide (compound 30); (xi) R₁ is H; R₂ is OH; and A is 3-azabicyclo[3.1.0]hexan-2,3-diyl, i.e., 2-(3-(2-amino-3-mercaptopropanoyl)-3-azabicyclo[3.1.0]hexane-2-carboxamido)-3-mercaptopropanoic acid (compound 31); (xii) R₁ is —COCH₃; R₂ is NH₂; and A is 3-azabicyclo[3.1.0]hexan-2,3-diyl, i.e., 3-(2-acetamido-3-mercaptopropanoyl)-N-(1-amino-3-mercapto-1-ox ° propan-2-yl)-3-azabicyclo[3.1.0]hexane-2-carboxamide (compound 32); (xiii) R₁ is H; R₂ is OH; and A is octahydrocyclopenta[b]pyrrole-1,2-diyl, i.e., 2-(1-(2-amino-3-mercapto propanoyl) octahydrocyclopenta[b]pyrrole-2-carboxamido)-3-mercaptopropanoic acid (compound 33); (xiv) R₁ is —COCH₃; R₂ is NH₂; and A is octahydrocyclopenta[b]pyrrole-1,2-diyl, i.e., 1-(2-acetamido-3-mercaptopropanoyl)-N-(1-amino-3-mercapto-1-oxopropan-2-yl)octahydro cyclopenta[b]pyrrole-2-carboxamide (compound 34); (xv) R₁ is H; R₂ is OH; and A is indoline-1,2-diyl, i.e., 2-(1-(2-amino-3-mercaptopropanoyl)indoline-2-carboxamido)-3-mercaptopropanoic acid (compound 35); or (xvi) R₁ is —COCH₃; R₂ is NH₂; and A is indoline-1,2-diyl, i.e., 1-(2-acetamido-3-mercaptopropanoyl)-N-(1-amino-3-mercapto-1-oxopropan-2-yl)indoline-2-carboxamide (compound 36).

The compounds of the general formula II may be synthesized according to any technology or procedure known in the art, e.g., as described in Example 6 and Scheme 1 hereinafter, starting from the N,N′-dicyclohexyl-carbodiimide (DCC) coupling reaction of N-Fmoc protected cyclic amino acids and the corresponding S-trityl protected cysteine analogues optionally comprising an ester moiety linked to the α-carbon, followed by hydrolysis of the ester moiety, if present, and deprotection of the protecting groups using, e.g., trifluoroacetic acid (TFA) and triethylsilane.

The compounds of the general formula II may have one or more asymmetric centers, and may accordingly exist both as enantiomers, i.e., optical isomers (R, S, or racemate, wherein a certain enantiomer may have an optical purity of 90%, 95%, 99% or more) and as diastereoisomers. Specifically, those chiral centers may be, e.g., in each one of the carbon atoms located at position alpha to any one of the carbonyl groups in the general formula II, as well as in each one of the carbon atoms of the ring A. It should be understood that according to the method of the present invention, inflammatory diseases of the lung caused by inhalation of toxics agent or irritants can be treated by administration of all such enantiomers, isomers and mixtures thereof, as well as pharmaceutically acceptable salts and solvates thereof.

TABLE 5
Compounds of the general formulas II, identified 21-36

| | |
|---|---|
| 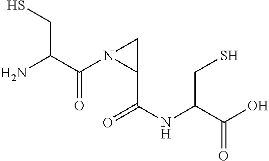 [SMILES: NC(CS)C(=O)N1CC1C(=O)NC(CS)C(=O)O] | 21 |
| 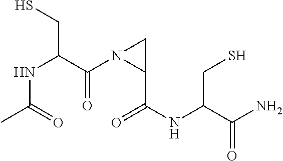 [SMILES: CC(=O)NC(CS)C(=O)N1CC1C(=O)NC(CS)C(N)=O] | 22 |
| 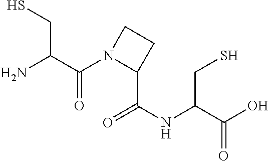 [SMILES: NC(CS)C(=O)N1CCC1C(=O)NC(CS)C(=O)O] | 23 |
| 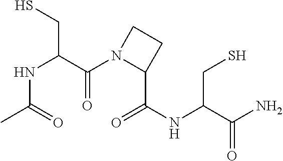 [SMILES: CC(=O)NC(CS)C(=O)N1CCC1C(=O)NC(CS)C(N)=O] | 24 |
| 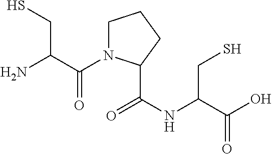 [SMILES: NC(CS)C(=O)N1CCCC1C(=O)NC(CS)C(=O)O] | 25 |
| 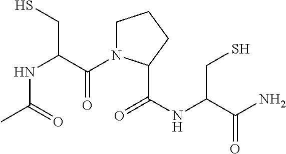 [SMILES: CC(=O)NC(CS)C(=O)N1CCCC1C(=O)NC(CS)C(N)=O] | 26 |
| 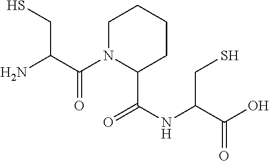 [SMILES: NC(CS)C(=O)N1CCCCC1C(=O)NC(CS)C(=O)O] | 27 |
| 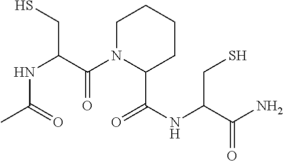 [SMILES: CC(=O)NC(CS)C(=O)N1CCCCC1C(=O)NC(CS)C(N)=O] | 28 |
| 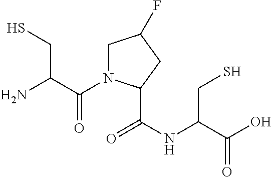 [SMILES: NC(CS)C(=O)N1CC(F)CC1C(=O)NC(CS)C(=O)O] | 29 |
| 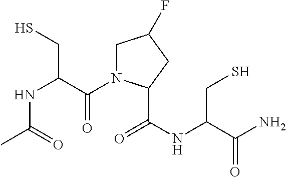 [SMILES: CC(=O)NC(CS)C(=O)N1CC(F)CC1C(=O)NC(CS)C(N)=O] | 30 |
| 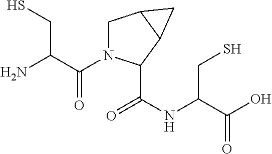 [SMILES: NC(CS)C(=O)N1CC2CC2C1C(=O)NC(CS)C(=O)O] | 31 |
| 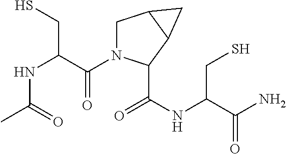 [SMILES: CC(=O)NC(CS)C(=O)N1CC2CC2C1C(=O)NC(CS)C(N)=O] | 32 |
| 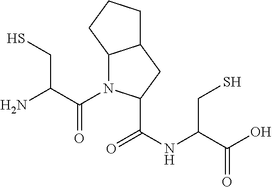 [SMILES: NC(CS)C(=O)N1C(C(=O)NC(CS)C(=O)O)CC2CCCC21] | 33 |
| 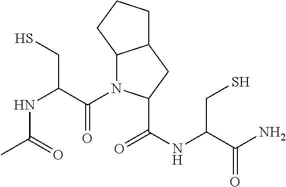 [SMILES: CC(=O)NC(CS)C(=O)N1C(C(=O)NC(CS)C(N)=O)CC2CCCC21] | 34 |
| 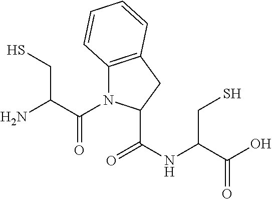 [SMILES: NC(CS)C(=O)N1c2ccccc2CC1C(=O)NC(CS)C(=O)O] | 35 |
| 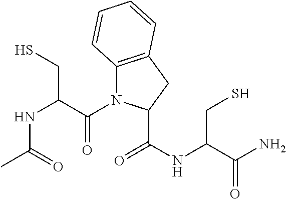 [SMILES: CC(=O)NC(CS)C(=O)N1c2ccccc2CC1C(=O)NC(CS)C(N)=O] | 36 |

Optically active forms of the compounds of the general formula II may be prepared using any method known in the art, e.g., by resolution of the racemic form by recrystallization techniques; by chiral synthesis; by extraction with chiral solvents; or by chromatographic separation using a chiral stationary phase. A non-limiting example of a method for obtaining optically active materials is transport across chiral membranes, i.e., a technique whereby a racemate is placed in contact with a thin membrane barrier, the concentration or pressure differential causes preferential transport across the membrane barrier, and separation occurs as a result of the non-racemic chiral nature of the membrane that allows only one enantiomer of the racemate to pass through. Chiral chromatography, including simulated moving bed chromatography, can also be used. A wide variety of chiral stationary phases are commercially available.

Cl₂ is the ninth largest produced chemical by volume in the United States, most of it being transported by rail to manufacturing plants (Evans, 2005). Current uses include pulp bleaching, waste sanitation, organic compound and pharmaceutical manufacturing, drinking water treatment, and maintenance of pathogen-free swimming pools (Leustik et al., 2008). Accidental or deliberate release of Cl₂ into the atmosphere has been associated with significant morbidity and mortality (Evans, 2005; Sexton and Pronchik, 1998). In addition, during the last few years, Cl₂ cylinders have been bundled with traditional explosives, raising significant concerns regarding the possible reemergence of this agent as a chemical weapon against both combatants and civilians (Bell, 2008).

A significant fraction of industrial accident victims exposed to 400 ppm Cl₂ developed pulmonary edema. A recent report also indicated that individuals exposed to Cl₂ in Iraq developed severe acute lung injury and required mechanical ventilation and supplemental oxygen to alleviate arterial hypoxemia (Bell, 2008). Currently, management of both animals and people exposed to Cl₂ consists of administration of supplemental oxygen to alleviate hypoxemia, β2 agonists and, corticosteroids to reverse bronchoconstriction and inflammation, and, in more severe cases, mechanical ventilation (Evans, 2005; Winder, 2001).

As shown in the Examples section hereinafter, R-100, R-907 and R-901 were found to be highly effective as a rescue therapy in a murine Cl₂ exposure model, According to the experimental protocol used, mice were exposed to 400 ppm Cl₂-containing air for a period of either 30 or 60 minutes, and were then administered 2 and 6 hours post exposure with a particular compound or combination of compounds. At 24 hours post-exposure to the Cl₂-containing air, blood samples were obtained from the inferior vena cava, the heart-lung block was rapidly excised, and the lungs were separated from the mediastinal tissues and were taken for biochemical assays and histological examination. All the compounds and combinations of compounds tested were found to ameliorate the effects of exposure to Cl₂ as exemplified by both lung morphology and biochemical markers. In another study, the effect of R-100 and R-907 on the survival and weight loss of the animals after a 60 minute exposure to 400 ppm Cl₂-containing air was examined, and as found, both compounds improved the survival of the animals, and reduced loss and improved recovery of body mass.

In certain particular embodiments, the methods of the present invention as defined above are used for treatment of an inflammatory disease of the lung caused by inhalation of Cl₂, i.e., for treatment of Cl₂ inhalational lung injury (CILI).

In other particular embodiments, the methods of the present invention as defined above are used for treatment of an inflammatory disease of the lung caused by inhalation of a toxic agent other than Cl₂, e.g., the chemical warfare agent phosgene or diphosgene, or an irritant such as smoke.

The term “treatment”, as used herein with respect to an inflammatory diseases of the lung caused by inhalation of a toxic agent or an irritant, refers to administration of a compound of the general formula I or II as defined above, or an enantiomer, diastereomer, racemate, or pharmaceutically acceptable salt or solvate thereof, after exposure to said toxic agent or irritant and following the onset of symptoms of said inflammatory disease, so as to ameliorate the effects of said toxic agent or irritant on the lungs. According to the invention, administration of said compound for treatment of CILI is aimed at reducing pulmonary edema and pulmonary shunt, diminishing PMN infiltration into the lung parenchyma, inhibiting a loss in pulmonary compliance, improving oxygenation, and decreasing carbon dioxide retention. The term “therapeutically effective amount” as used herein refers to the quantity of said compound that is useful to treat said inflammatory disease in general, or CILI in particular.

### Pharmaceutical Compositions for Treatment of CILI

In A further aspect, the present invention provides a pharmaceutical composition for treatment of an inflammatory disease of the lung caused by inhalation of a toxic agent or an irritant, said composition comprising a pharmaceutically acceptable carrier and an active agent, more particularly a compound of the general formula I or II as defined above, or an enantiomer, diastereomer, racemate, or pharmaceutically acceptable salt or solvate thereof. In certain embodiments, the active agent is R-100, R-907, R-901, or an enantiomer, diastereomer, racemate, or pharmaceutically acceptable salt or solvate of any one of the aforesaid.

The pharmaceutical compositions of the present invention can be provided in a variety of formulations, e.g., in a pharmaceutically acceptable form and/or in a salt form, as well as in a variety of dosages.

In one embodiment, the pharmaceutical composition of the present invention comprises a non-toxic pharmaceutically acceptable salt of the active agent as defined above. Suitable pharmaceutically acceptable salts include acid addition salts such as, without being limited to, those formed with hydrochloric acid, fumaric acid, p-toluenesulfonic acid, maleic acid, succinic acid, acetic acid, citric acid, tartaric acid, carbonic acid, or phosphoric acid. Salts of amine groups may also comprise quaternary ammonium salts in which the amino nitrogen atom carries a suitable organic group such as an alkyl, alkenyl, alkynyl, or aralkyl moiety. Furthermore, where an active agent carry an acidic moiety, suitable pharmaceutically acceptable salts thereof may include metal salts such as alkali metal salts, e.g., sodium or potassium salts, and alkaline earth metal salts, e.g., calcium or magnesium salts.

The pharmaceutically acceptable salts of the present invention may be formed by conventional means, e.g., by reacting the free base form of the active agent with one or more equivalents of the appropriate acid in a solvent or medium in which the salt is insoluble, or in a solvent such as water which is removed in vacuo or by freeze drying, or by exchanging the anions of an existing salt for another anion on a suitable ion exchange resin.

The present invention encompasses solvates of the various active agents defined above as well as salts thereof, e.g., hydrates.

The pharmaceutical compositions provided by the present invention may be prepared by conventional techniques, e.g., as described in Remington: The Science and Practice of Pharmacy, 19^{th} Ed., 1995. The compositions can be prepared, e.g., by uniformly and intimately bringing the active agent, i.e., the compound of the general formula I or II, into association with a liquid carrier, a finely divided solid carrier, or both, and then, if necessary, shaping the product into the desired formulation. The compositions may be in liquid, solid or semisolid form and may further include pharmaceutically acceptable fillers, carriers, diluents or adjuvants, and other inert ingredients and excipients. In one embodiment, the pharmaceutical composition of the present invention is formulated as nanoparticles.

The compositions can be formulated for any suitable route of administration, but they are preferably formulated for parenteral administration, e.g., intravenous, intraarterial, intramuscular, intraperitoneal, intrathecal, intrapleural, subcutaneous, intratracheal or administration, as well as for inhalation. The dosage will depend on the state of the patient, and will be determined as deemed appropriate by the practitioner.

The pharmaceutical composition of the invention may be in the form of a sterile injectable aqueous or oleagenous suspension, which may be formulated according to the known art using suitable dispersing, wetting or suspending agents. The sterile injectable preparation may also be a sterile injectable solution or suspension in a non-toxic parenterally acceptable diluent or solvent. Acceptable vehicles and solvents that may be employed include, without limiting, water, Ringer's solution and isotonic sodium chloride solution.

Pharmaceutical compositions according to the present invention, when formulated for inhalation, may be administered utilizing any suitable device known in the art, such as metered dose inhalers, liquid nebulizers, dry powder inhalers, sprayers, thermal vaporizers, electrohydrodynamic aerosolizers, and the like.

Pharmaceutical compositions according to the present invention, when formulated for administration route other than parenteral administration, may be in a form suitable for oral use, e.g., as tablets, troches, lozenges, aqueous, or oily suspensions, dispersible powders or granules, emulsions, hard or soft capsules, or syrups or elixirs. Compositions intended for oral use may be prepared according to any method known to the art for the manufacture of pharmaceutical compositions and may further comprise one or more agents selected from sweetening agents, flavoring agents, coloring agents and preserving agents in order to provide pharmaceutically elegant and palatable preparations. Tablets contain the active agent(s) in admixture with non-toxic pharmaceutically acceptable excipients, which are suitable for the manufacture of tablets. These excipients may be, e.g., inert diluents such as calcium carbonate, sodium carbonate, lactose, calcium phosphate, or sodium phosphate; granulating and disintegrating agents, e.g., corn starch or alginic acid; binding agents, e.g., starch, gelatin or acacia; and lubricating agents, e.g., magnesium stearate, stearic acid, or talc. The tablets may be either uncoated or coated utilizing known techniques to delay disintegration and absorption in the gastrointestinal tract and thereby provide a sustained action over a longer period. For example, a time delay material such as glyceryl monostearate or glyceryl distearate may be employed. They may also be coated using the techniques described in the U.S. Pat. Nos. 4,256,108, 4,166,452 and 4,265,874 to form osmotic therapeutic tablets for control release. The pharmaceutical composition of the invention may also be in the form of oil-in-water emulsion.

The pharmaceutical compositions of the invention may be formulated for controlled release of the active agent. Such compositions may be formulated as controlled-release matrix, e.g., as controlled-release matrix tablets in which the release of a soluble active agent is controlled by having the active diffuse through a gel formed after the swelling of a hydrophilic polymer brought into contact with dissolving liquid (in vitro) or gastro-intestinal fluid (in vivo). Many polymers have been described as capable of forming such gel, e.g., derivatives of cellulose, in particular the cellulose ethers such as hydroxypropyl cellulose, hydroxymethyl cellulose, methylcellulose or methyl hydroxypropyl cellulose, and among the different commercial grades of these ethers are those showing fairly high viscosity. In other configurations, the compositions comprise the active agent formulated for controlled release in microencapsulated dosage form, in which small droplets of the active agent are surrounded by a coating or a membrane to form particles in the range of a few micrometers to a few millimeters.

Another contemplated formulation is depot systems, based on biodegradable polymers, wherein as the polymer degrades, the active agent is slowly released. The most common class of biodegradable polymers is the hydrolytically labile polyesters prepared from lactic acid, glycolic acid, or combinations of these two molecules. Polymers prepared from these individual monomers include poly (D,L-lactide) (PLA), poly (glycolide) (PGA), and the copolymer poly (D,L-lactide-co-glycolide) (PLG).

In yet a further aspect, the present invention provides a compound of the general formula I or II as defined above, or an enantiomer, diastereomer, racemate, or pharmaceutically acceptable salt or solvate thereof, for use in treatment of an inflammatory disease of the lung caused by inhalation of a toxic agent or an irritant.

In still a further aspect, the present invention relates to use of a compound of the general formula I or II as defined above, or an enantiomer, diastereomer, racemate, or pharmaceutically acceptable salt or solvate thereof, for the preparation of a pharmaceutical composition for treatment of an inflammatory disease of the lung caused by inhalation of a toxic agent or an irritant.

In yet another aspect, the present invention provides a compound of the general formula II as defined above, or an enantiomer, diastereomer, racemate, or pharmaceutically acceptable salt or solvate thereof, but excluding the compounds wherein R₁ is H or —COCH₃, R₂ is OH or NH₂, and A is pyrrolidin-1,2-diyl.

In certain embodiments, the compound of the present invention is a compound of the general formula II, wherein R₁ is H, —CO(C₁-C₄)alkyl, preferably —COCH₃ or —COCH₂CH₃, —COO(C₁-C₄)alkyl, preferably —COOCH₃ or —COOCH₂CH₃, or —CONH(C₁-C₄)alkyl, preferably —CONHCH₃ or —CONHCH₂CH₃.

In certain embodiments, the compound of the present invention is a compound of the general formula II, wherein R₂ is —OH, or N(R₃R₄), wherein R₃ and R₄ each independently is H, or (C₁-C₄)alkyl, preferably methyl or ethyl.

In certain embodiments, the compound of the present invention is a compound of the general formula II, wherein A is a 3-, 4-, 5-, or 6-membered ring, wherein each one of the carbon atoms in said ring may be substituted by oxo, H, halogen, (C₁-C₄)alkyl, NO₂, N(R₅R₆), —OR₅, —SR₅, —SO₂R₅, or —COR₇, or two adjacent carbon atoms in said ring form a 3-6 membered saturated, partially saturated, or aromatic carbocyclic or heterocyclic ring; R₅ and R₆ each independently is H, or (C₁-C₄)alkyl; and R₇ is OH, NH₂, or —O(C₁-C₄)alkyl. In particular such embodiments, A is a 3-6 membered ring, wherein each one of the carbon atoms in said ring may be substituted by oxo, H, halogen, methyl, ethyl, NO₂, —NH₂, OH, —OCH₃, —OCH₂CH₃, —SH, —SCH₃, —SCH₂CH₃, —SO₂H, —SO₂CH₃, —SO₂CH₂CH₃, —COOH, —COOCH₃, —COOCH₂CH₃, or —CONH₂, or two adjacent carbon atoms in said ring form a 3-6 membered saturated, partially saturated, or aromatic carbocyclic or heterocyclic ring.

In particular embodiments, the compound of the present invention is a compound of the general formula II as defined above, wherein R₁ is H, or —CO(C₁-C₄)alkyl; R₂ is —OH, or N(R₃R₄), wherein R₃ and R₄ each independently is H, or (C₁-C₄)alkyl; A is a 3-6 membered ring, wherein each one of the carbon atoms in said ring may be substituted by oxo, H, halogen, (C₁-C₄)alkyl, NO₂, N(R₅R₆), —OR₅, —SR₅, —SO₂R₅, or —COR₇, or two adjacent carbon atoms in said ring form a 3-6 membered saturated, partially saturated, or aromatic carbocyclic or heterocyclic ring; R₅ and R₆ each independently is H, methyl or ethyl; and R₇ is OH, NH₂, methoxy or ethoxy. More particular such embodiments are those wherein R₁ is H, —COCH₃, or —COCH₂CH₃; R₂ is OH or N(R₃R₄), wherein R₃ and R₄ each independently is H, methyl, or ethyl; and A is a 3-6 membered ring, wherein each one of the carbon atoms in said ring may be substituted by oxo, H, halogen, methyl, ethyl, NO₂, —NH₂, OH, —OCH₃, —OCH₂CH₃, —SH, —SCH₃, —SCH₂CH₃, —SO₂H, —SO₂CH₃, —SO₂CH₂CH₃, —COOH, —COOCH₃, —COOCH₂CH₃, or —CONH₂, or two adjacent carbon atoms in said ring form a 3-6 membered saturated, partially saturated, or aromatic carbocyclic or heterocyclic ring. Most particular such embodiments are those wherein R₁ is H or —COCH₃; R₂ is OH or NH₂; and A is azeridin-diyl, azetidin-1,2-diyl, pyrrolidin-1,2-diyl, or piperidin-1,2-diyl, wherein each one of the carbon atoms in said ring may be substituted by halogen, or two adjacent carbon atoms in said ring form cyclopropane, cyclobutane, cyclopentane, cyclohexane, or benzene.

Specific compounds of the general formulas II described herein and encompassed by the present invention are herein identified compounds 21-24 and 27-36, and their full chemical structures are depicted in Table 5 above.

In still another aspect, the present invention provides a pharmaceutical composition comprising a compound of the general formula II as defined above, or an enantiomer, diastereomer, racemate, or pharmaceutically acceptable salt or solvate thereof, but excluding the compounds wherein R₁ is H or —COCH₃, R₂ is OH or NH₂, and A is pyrrolidin-1,2-diyl, and a pharmaceutically acceptable carrier.

The invention will now be illustrated by the following non-limiting Examples.

### Examples

### Example 1

### R-100 is Effective as a Rescue Therapy in a Murine Cl₂ Exposure Model

In this study, the therapeutic effect of 3-nitratomethyl-2,2,5,5-tetramethylpyrrolidinyloxy(R-100), a 1-pyrrolidinyloxy derivative of the general formula I, in treatment of CILI was tested.

In a chemical fume hood, Balb/c mice (n=4 in each group) were exposed in a cylindrical glass chamber (4 mice per exposure) that is flushed continuously for 30 minutes at a rate of 2 liters/minute with humidified gas obtained from a calibrated cylinder containing air and 400 ppm Cl₂. After the end of the 30 minute exposure, the chamber was opened and mice were removed and placed immediately in cages in room air. Two and six hours after the conclusion of Cl₂ exposure, mice were administered intraperitoneal (IP) with various concentrations (4, 12, 40, and 80 mg/kg/dose) of R-100. As this compound is poorly soluble in water, a stable aqueous solution was prepared by formulating the compound in hydroxypropylcyclodextrin (HPCD). HPCD alone was used as a control. At 24 hours post-exposure to the Cl₂-containing air, a midline incision from the neck to the pubis was created for access to the chest and abdominal cavities. Blood samples were obtained from the inferior vena cava just before sacrifice, the heart-lung block was rapidly excised, and the pulmonary circulation was flushed through the main pulmonary artery with 20 ml of normal saline. The lungs were separated from the mediastinal tissues and were taken for biochemical assays and histological examination (H&E staining). The following morphological criteria were used for scoring: 0, normal lung; grade 1, minimal edema or infiltration of alveolar or bronchiolar walls; grade 3, moderate edema and inflammatory cell infiltration without obvious damage to lung architecture; grade 4, severe inflammatory cell infiltration with obvious damage to lung architecture.

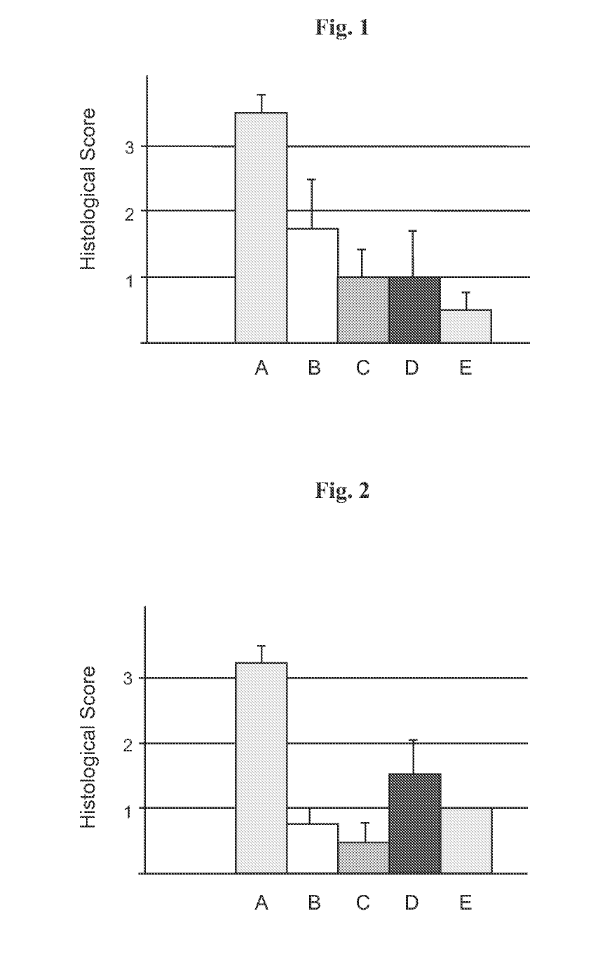

As shown in FIG. 1, R-100, when administered 2 and 6 hours post a 30 minute exposure to Cl₂-containing air, dose-dependently attenuated CILI in mice 24 hours post exposure as exemplified by the improved histology scores.

### Example 2

### R-907 is Effective as a Rescue Therapy in a Murine Cl₂ Exposure Model

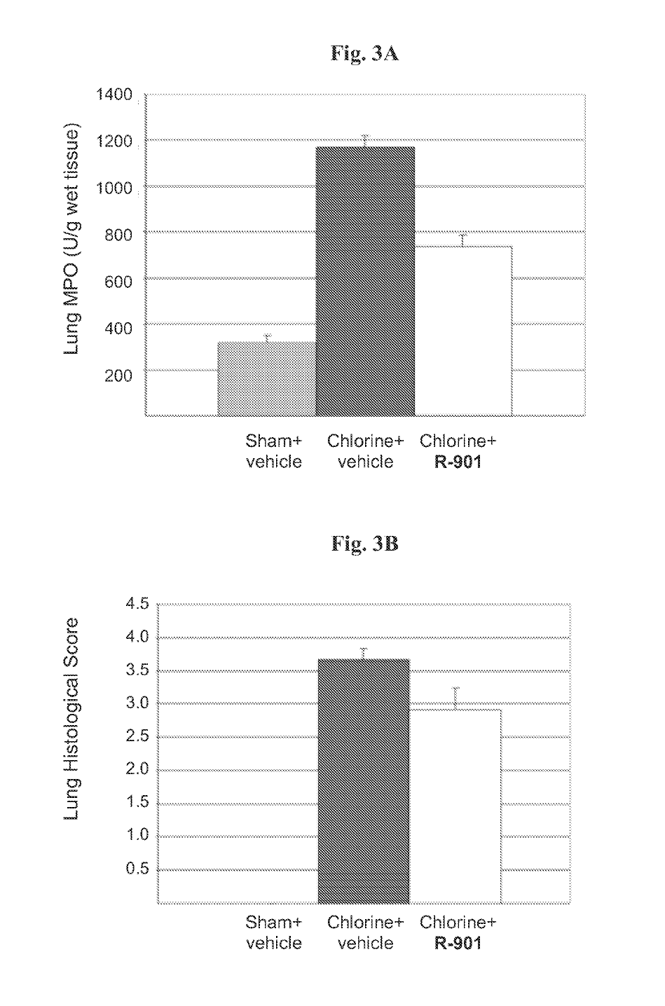

In this study, the therapeutic effect of 2-(1-(2-amino-3-mercaptopropanoyl) pyrrolidine-2-carboxamido)-3-mercaptopropanoic acid (R-907), a compound of the general formula II representing the amino-acid sequence Cys-Pro-Cys, in treatment of CILI was tested, using the experimental protocol described in Example 1. The active agent was formulated saline and administered 2 and 6 hours after the conclusion of Cl₂ exposure at concentrations of 3, 10, 30, and 80 mg/kg/dose). Saline alone was used as a control. FIG. 2 shows that R-907, when administered 2 and 6 hours post a 30 minute exposure to Cl₂-containing air, dose-dependently attenuated CILI in mice 24 hours post exposure as exemplified by the improved histology scores.

### Example 3

### R-901 is Effective as a Rescue Therapy in a Murine Cl₂ Exposure Model

In this study, the therapeutic effect of 1-(2-acetamido-3-mercapto propanoyl)-N-(1-amino-3-mercapto-1-oxopropan-2-yl)pyrrolidine-2-carboxamide (R-901), a compound of the general formula II representing the amino-acid sequence Cys-Pro-Cys in which the terminal amino group is acylated and the terminal carboxyl group is amidated, in treatment of CILI was tested.

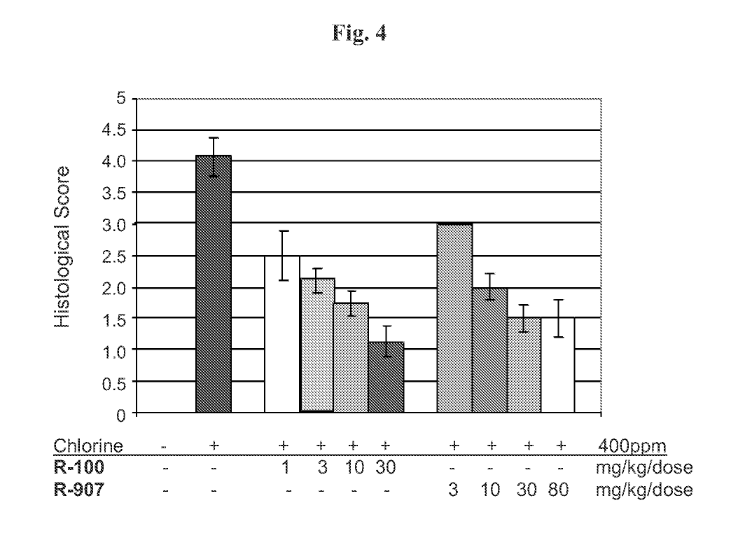

Male Balb/c mice (25 g) were exposed in a closed environmental chamber to 400 ppm Cl₂ in air for 30 minutes. 15 minutes after the conclusion of Cl₂ exposure, mice were initiated on a 12 hourly regimen of R-901 (30 mg/kg/dose IP in 0.5 ml dextrose in water [D5W]). At 24 hours, mice were euthanized and lung tissue was taken for examination of PMN infiltration, as reflected by MPO level, and histology (H&E staining, by a pathologist blinded to experimental group assignment). FIG. 3 shows that R-901 therapy reduced the elevation in pulmonary MPO (3A) and histological damage (3B) by 50% (p<0.0001) and 20% (n.s.), respectively, relative to placebo (D5W).

### Example 4

### The Therapeutic Effect of R-100 and R-907 in a Severe CILI Model

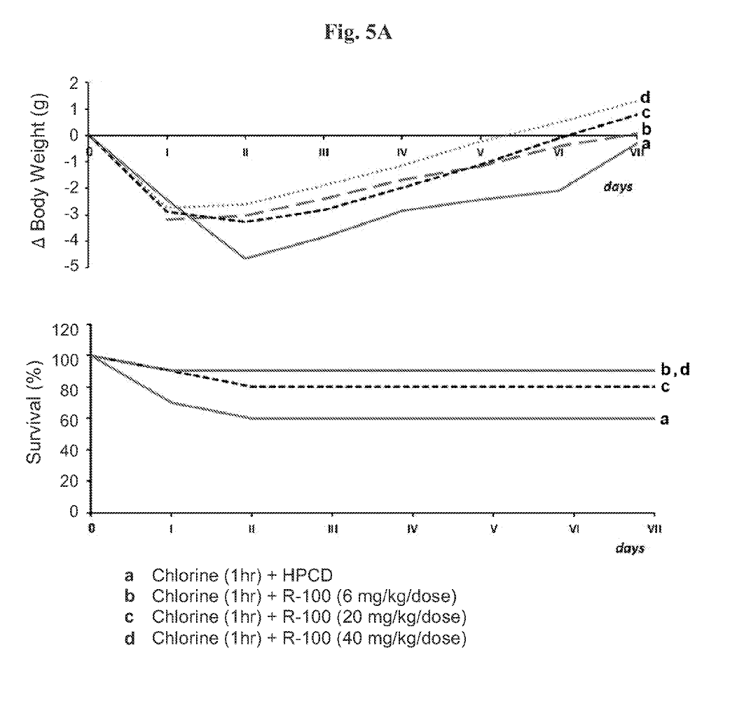

In this study, the therapeutic effects of R-100 and R-907 were tested in a severe CILI model, in which animal were exposed to 400 ppm Cl₂-containing air as described in Example 1 for 60 minutes instead of 30 minutes. FIG. 4 shows that both R-100 and R-907, when administered 2 and 6 hours post a 60 minute exposure to Cl₂-containing air at concentrations of 1, 3, 10, or 30 mg/kg/dose (R-100) and 3, 10, 30, or 80 mg/kg/dose (R-907), attenuated CILI in mice 24 hours post exposure as exemplified by the improved histology scores.

### Example 5

### Survival Studies after Exposure to a Severe CILI Model

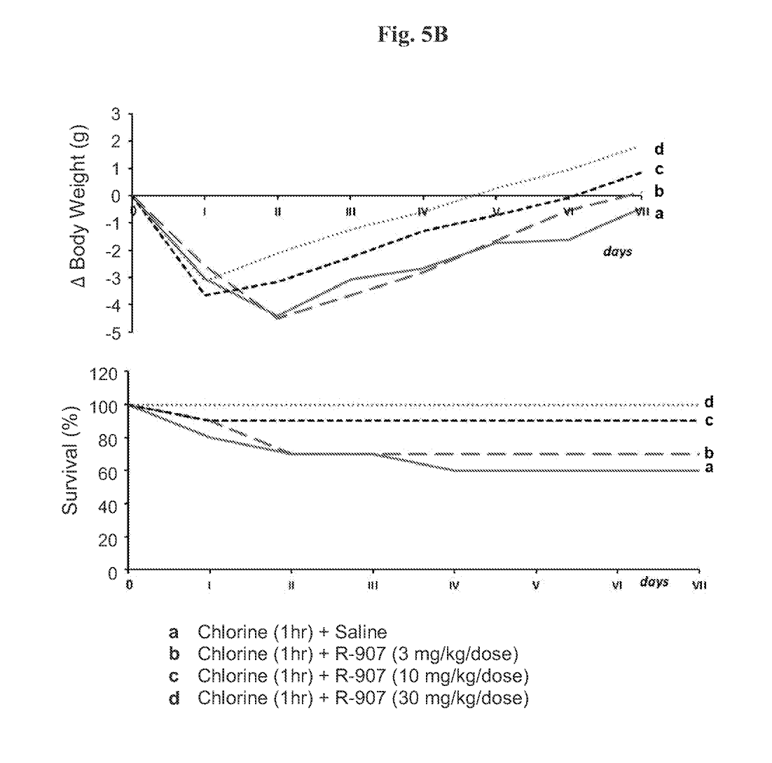

In this study, the therapeutic effects of R-100 and R-907, when administered 2 and 6 hours post a 60 minute exposure to Cl₂-containing air, on the survival of the animal (n=10 in each group) as well as their weight loss, was tested. R-100 was formulated in HPCD and was administered at a concentration of 6, 20 or 40 mg/kg/dose; and R-907 was formulated in saline and was administered at a concentration of 3, 10 or 30 mg/kg/dose. FIGS. 5A and 5B show the beneficial effects of R-100 and R-907, respectively, on the survival and weight loss of the animals after a 60 minute exposure to 400 ppm Cl₂-containing air. As shown in these figures, both compounds improved the survival of the animals, and reduced loss and improved recovery of body mass.

### Example 6

### Synthesis of Compounds of the General Formula II

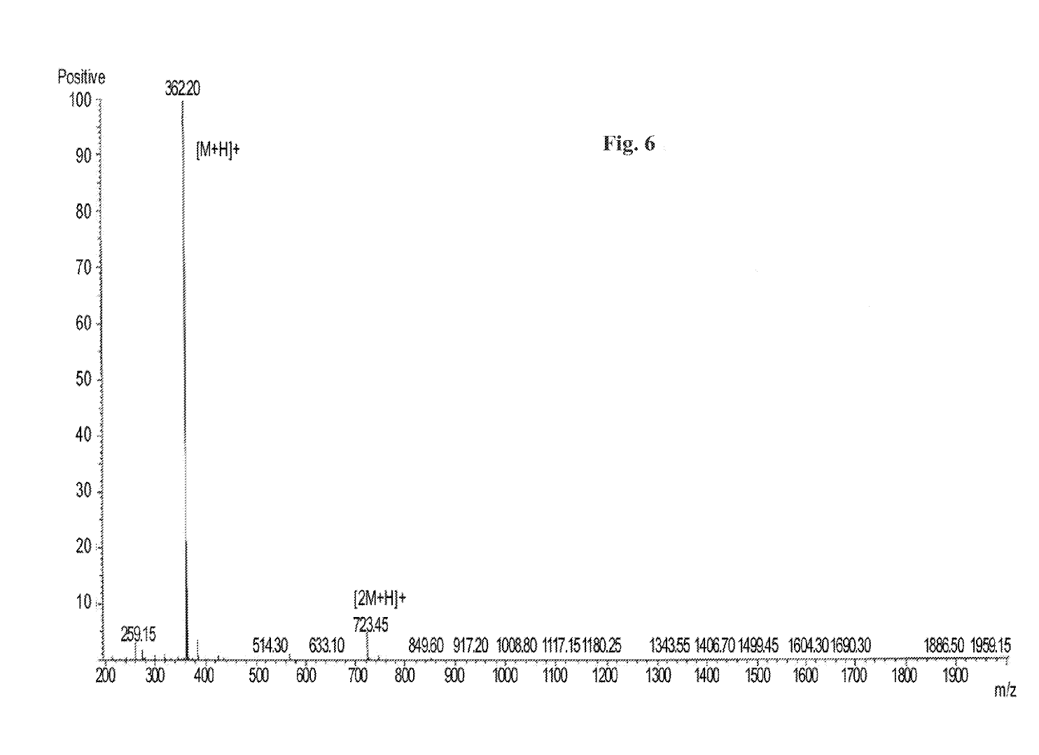

Scheme 1 depicts a procedure for the syntheses of compounds 33 and 34, representing compounds of the general formula II. As shown in this Scheme, compounds 33 and 34 are synthesized from the DCC coupling reaction of N-Fmoc protected octahydrocyclopenta[b]pyrrole-2-carboxylic acid and the corresponding S-trityl protected cysteine analogues. The protecting groups are then removed by hydrolysis, if required, followed by deprotection of t-Boc and S-trityl protecting groups using TFA and triethylsilane. FIG. 6 shows the mass spectrometry data of compound 33 prepared according to the procedure described (MS (ES⁺): m/z 362.20 (M+1)), which matches with the desired structure. As shown in the generic synthetic approach depicted in Scheme 2, other compounds of the general formula II such as compounds 21-32 and 35-36 can be synthesized from the corresponding N-Fmoc protected cyclic amino acids following the synthetic approach shown in Scheme 1.

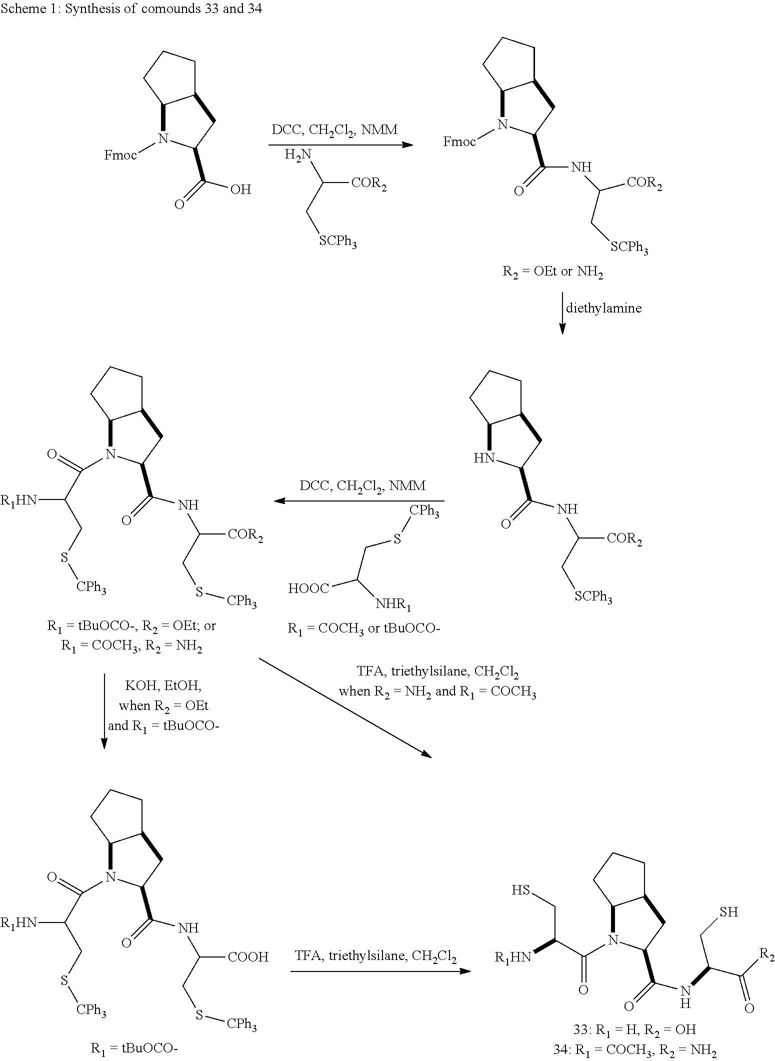

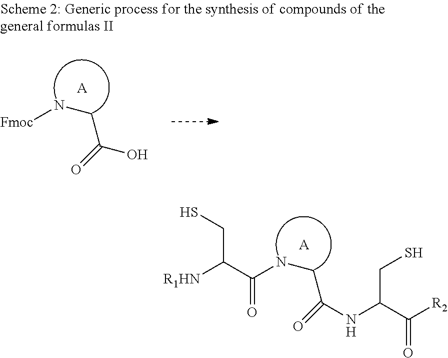

### REFERENCES

Batchinsky, A. I., Martini, D. K., Jordan, B. S., Dick, E. J., Fudge, J., Baird, C. A., Hardin, D. E., Cancio, L. C., Acute respiratory distress syndrome secondary to inhalation of chlorine gas in sheep. J Trauma, 2006, 60(5), 944-956; discussion 956-957
Bell, D. G., Management of acute respiratory distress syndrome (ARDS) following chlorine exposure (Abstract), Am J Respir Crit Care Med., 2008, 176, A314
Evans, R. B., Chlorine: state of the art, Lung, 2005, 183, 151-167
Guo, Y., Krumwiede, M., White, J. G., Wangensteen, O. D., HOCl effects on the tight junctions of rabbit tracheal epithelium. Am J Physiol., 1996, 270(2 Pt 1), L224-31
Hoshino, T., Nakamura, H., Okamoto, M., Kato, S., Araya, S., Nomiyama, K., Oizumi, K., Young, H. A., Aizawa, H., Yodoi, J., Redox-active protein thioredoxin prevents proinflammatory cytokine- or bleomycin-induced lung injury. Am J Respir Crit Care Med., 2003, 168(9), 1075-1083
Hoyle, G. W., Mitigation of chlorine lung injury by increasing cyclic AMP levels. Proc Am Thorac Soc., 2010, 7(4), 284-289
Leustik, M., Doran, S., Bracher. A., Williams, S., Squadrito. Schoeb, T. R., Postlethwait, E. Matalon, S., Mitigation of chlorine-induced lung injury by low-molecular weight antioxidants. Am J Physiol Lung Cell Mol Physiol., 2008, 295(5), L733-743
Sexton, J. D., Pronchik, D. J., Chlorine inhalation: the big picture. J Toxicol Clin Toxicol., 1998, 36, 87-93
Squadrito, G. L., Postlethwait, E. M., Matalon, S., Elucidating mechanisms of chlorine toxicity: reaction kinetics, thermodynamics, and physiological implications. Am J Physiol Lung Cell Mol Physiol., 2010, 299(3), L289-300
Tian, X., Tao, H., Brisolara, J., Chen, J., Rando, R. J., Hoyle, G. W., Acute lung injury induced by chlorine inhalation in C57BL/6 and FVB/N mice. Inhal Toxicol., 2008, 20(9), 783-793
Tipple, T. E., Welty, S. E., Rogers, L. K., Hansen, T. N., Choi, Y. E., Kehrer, J. P., Smith, C. V., Thioredoxin-related mechanisms in hyperoxic lung injury in mice. Am J Respir Cell Mol Biol., 2007, 37(4), 405-413
Winder, C., The toxicology of chlorine. Environ Res., 2001, 85, 105-114
Yadav, A. K., Bracher, A., Doran, S. F., Leustik, M., Squadrito, G. L., Postlethwait, E. M., Matalon, S., Mechanisms and modification of chlorine-induced lung injury in animals. Proc Am Thorac Soc., 2010, 7(4), 278-283

1. A compound of the general formula II: 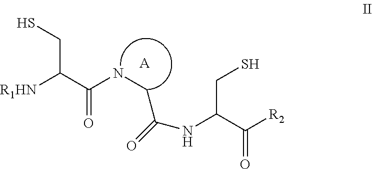
or an enantiomer, diastereomer, racemate, or a pharmaceutically acceptable salt or solvate thereof,
wherein
R₁ is H, —CO(C₁-C₈)alkyl, —COO(C₁-C₈)alkyl or —CONH(C₁-C₈)alkyl;
R₂ is OH, or N(R₃R₄);
R₃ and R₄ each independently is H, (C₁-C₈)alkyl, (C₃-C₁₀)cycloalkyl, 4-12-membered heterocyclyl, or (C₆-C₁₄)aryl;
A is a 3, 4, 5 or 6 membered ring optionally containing one or more additional heteroatoms selected from the group consisting of sulfur, oxygen or nitrogen, wherein said nitrogen atom may be substituted by (C₁-C₈)alkyl, and each one of the carbon atoms in said ring may be substituted by oxo, halogen, (C₁-C₈)alkyl, (C₆-C₁₄)aryl, 4-12-membered heterocyclyl, NO₂, N(R₅R₆), —OR₅, —SR₅, —SO₂R₅, or —COR₇, or two adjacent carbon atoms in said 3, 4, or 5 membered ring form a 3-6 membered saturated, partially saturated, or aromatic carbocyclic or heterocyclic ring, or two adjacent carbon atoms in said 6 membered ring form a 3-6 membered saturated, or partially saturated carbocyclic or heterocyclic ring;
R₅ and R₆ each independently is H, or (C₁-C₈)alkyl; and
R₇ is OH, NH₂, or —O(C₁-C₈)alkyl,
but excluding the compounds wherein R₁ is H or —COCH₃, R₂ is OH or NH₂, and A is pyrrolidin-1,2-diyl.

2. The compound of claim 1, wherein R₁ is H, —CO(C₁-C₄)alkyl, —COO(C₁-C₄)alkyl, or —CONH(C₁-C₄)alkyl.

3. The compound of claim 1, wherein R₂ is —OH, or N(R₃R₄), wherein R₃ and R₄ each independently is H, or (C₁-C₄)alkyl.

4. The compound of claim 1, wherein A is a 3, 4, 5 or 6 membered ring, wherein each one of the carbon atoms in said ring may be substituted by oxo, H, halogen, (C₁-C₄)alkyl, NO₂, N(R₅R₆), —OR₅, —SR₅, —SO₂R₅, or —COR₇, or two adjacent carbon atoms in said 3, 4, or 5 membered ring form a 3-6 membered saturated, partially saturated, or aromatic carbocyclic or heterocyclic ring, or two adjacent carbon atoms in said 6 membered ring form a 3-6 membered saturated, or partially saturated carbocyclic or heterocyclic ring; R₅ and R₆ each independently is H, or (C₁-C₄)alkyl; and R₇ is OH, NH₂, or —O(C₁-C₄)alkyl.

5. The compound of claim 4, wherein A is a 3, 4, 5 or 6 membered ring, wherein each one of the carbon atoms in said ring may be substituted by oxo, H, halogen, methyl, ethyl, NO₂, —NH₂, OH, —OCH₃, —OCH₂CH₃, —SH, —SCH₃, —SCH₂CH₃, —SO₂H, —SO₂CH₃, —SO₂CH₂CH₃, —COOH, —COOCH₃, —COOCH₂CH₃, or —CONH₂, or two adjacent carbon atoms in said 3, 4 or 5 membered ring form a 3-6 membered saturated, partially saturated, or aromatic carbocyclic or heterocyclic ring, or two adjacent carbon atoms in said 6 membered ring form a 3-6 membered saturated, or partially saturated carbocyclic or heterocyclic ring.

6. The compound of claim 1, wherein:
(i) R₁ is H, or —CO(C₁-C₄)alkyl;
(ii) R₂ is —OH, or N(R₃R₄), wherein R₃ and R₄ each independently is H, or (C₁-C₄)alkyl;
(iii) A is a 3, 4, 5 or 6 membered ring, wherein each one of the carbon atoms in said ring may be substituted by oxo, H, halogen, (C₁-C₄)alkyl, NO₂, N(R₅R₆), —OR₅, —SR₅, —SO₂R₅, or —COR₇, or two adjacent carbon atoms in said 3, 4 or 5 membered ring form a 3-6 membered saturated, partially saturated, or aromatic carbocyclic or heterocyclic ring, or two adjacent carbon atoms in said 6 membered ring form a 3-6 membered saturated, or partially saturated carbocyclic or heterocyclic ring;
(iv) R₅ and R₆ each independently is H, methyl or ethyl; and
(v) R₇ is OH, NH₂, methoxy or ethoxy.

7. The compound of claim 6, wherein:
(i) R₁ is H, —COCH₃, or —COCH₂CH₃;
(ii) R₂ is OH or N(R₃R₄), wherein R₃ and R₄ each independently is H, methyl, or ethyl; and
(iii) A is a 3, 4, 5 or 6 membered ring, wherein each one of the carbon atoms in said ring may be substituted by oxo, H, halogen, methyl, ethyl, NO₂, —NH₂, OH, —OCH₃, —OCH₂CH₃, —SH, —SCH₃, —SCH₂CH₃, —SO₂H, —SO₂CH₃, —SO₂CH₂CH₃, —COOH, —COOCH₃, —COOCH₂CH₃, or —CONH₂, or two adjacent carbon atoms in said 3, 4, or 5 membered ring form a 3-6 membered saturated, partially saturated, or aromatic carbocyclic or heterocyclic ring, or two adjacent carbon atoms in said 6 membered ring form a 3-6 membered saturated, or partially saturated carbocyclic or heterocyclic ring.

8. The compound of claim 7, wherein:
(i) R₁ is H or —COCH₃;
(ii) R₂ is OH or NH₂; and
(iii) A is azeridin-diyl, azetidin-1,2-diyl, pyrrolidin-1,2-diyl, or piperidin-1,2-diyl, wherein each one of the carbon atoms in said ring may be substituted by halogen, or two adjacent carbon atoms in said azeridin-diyl, azetidin-1,2-diyl or pyrrolidin-1,2-diyl form cyclopropane, cyclobutane, cyclopentane, cyclohexane, or benzene, or two adjacent carbon atoms in said piperidin-1,2-diyl form cyclopropane, cyclobutane, cyclopentane or cyclohexane.

9. The compound of claim 8, wherein:
(i) R₁ is H; R₂ is OH; and A is azeridin-diyl, herein identified compound 21;
(ii) R₁ is —COCH₃; R₂ is NH₂; and A is azeridin-diyl, herein identified compound 22;
(iii) R₁ is H; R₂ is OH; and A is azetidin-1,2-diyl, herein identified compound 23;
(iv) R₁ is —COCH₃; R₂ is NH₂; and A is azetidin-1,2-diyl, herein identified compound 24;
(v) R₁ is H; R₂ is OH; and A is piperidin-1,2-diyl, herein identified compound 27;
(vi) R₁ is —COCH₃; R₂ is NH₂; and A is piperidin-1,2-diyl, herein identified compound 28;
(vii) R₁ is H; R₂ is OH; and A is 4-fluoropyrrolidin-1,2-diyl, herein identified compound 29;
(viii) R₁ is —COCH₃; R₂ is NH₂; and A is 4-fluoropyrrolidin-1,2-diyl, herein identified compound 30;
(ix) R₁ is H; R₂ is OH; and A is 3-azabicyclo[3.1.0]hexan-2,3-diyl, herein identified compound 31;
(x) R₁ is —COCH₃; R₂ is NH₂; and A is 3-azabicyclo[3.1.0]hexan-2,3-diyl, herein identified compound 32;
(xi) R₁ is H; R₂ is OH; and A is octahydrocyclopenta[b]pyrrole-1,2-diyl, herein identified compound 33;
(xii) R₁ is —COCH₃; R₂ is NH₂; and A is octahydrocyclopenta[b]pyrrole-1,2-diyl, herein identified compound 34);
(xiii) R₁ is H; R₂ is OH; and A is indoline-1,2-diyl, herein identified compound 35; or
(xiv) R₁ is —COCH₃; R₂ is NH₂; and A is indoline-1,2-diyl, herein identified compound 36.

10. A pharmaceutical composition comprising a compound of the general formula II as claimed in claim 1, or an enantiomer, diastereomer, racemate, or a pharmaceutically acceptable salt or solvate thereof, and a pharmaceutically acceptable carrier.

11. A method for treatment of an inflammatory disease of the lung caused by inhalation of a toxic agent selected from the group consisting of chlorine, phosgene and diphosgene, in an individual in need thereof, said method comprising administering to said individual a therapeutically effective amount of a compound according to claim 1.

12.-19. (canceled)

20. The method of claim 11, comprising administering a compound of the general formula II, wherein (i) R₁ is H; R₂ is OH; and A is pyrolidin-1,2-diyl; or (ii) R₁ is —COCH₃; R₂ is NH₂; and A is pyrolidin-1,2-diyl, or an enantiomer, diastereomer, racemate, or pharmaceutically acceptable salt or solvate thereof.

21. A method for treatment of an inflammatory disease of the lung caused by inhalation of a toxic agent or an irritant, in an individual in need thereof, said method comprising administering to said individual a therapeutically effective amount of a compound of the general formula I: 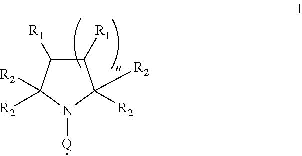
or an enantiomer, diastereomer, racemate, or pharmaceutically acceptable salt or solvate thereof,
wherein
R₁ each independently is H, —OH, —COR₃, —COOR₃, —OCOOR₃, —OCON(R₃)₂, —(C₁-C₁₆)alkylene-COOR₃, —CN, —NO₂, —SH, —SR₃, —(C₁-C₁₆)alkyl, —O—(C₁-C₁₆)alkyl, —N(R₃)₂, —CON(R₃)₂, —SO₂R₃, —S(═O)R₃, or a nitric oxide donor group of the formula —X₁-X₂-X₃, wherein X₁ is absent or selected from the group consisting of —O—, —S— or —NH—; X₂ is absent or is (C₁-C₂₀)alkylene optionally substituted by one or more —ONO₂ groups and optionally further substituted by a moiety of the general formula D: 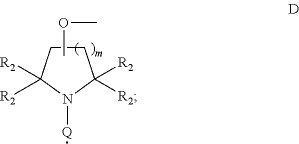
and X₃ is —NO or —ONO₂, provided that at least one R₁ group is a nitric oxide donor group;
R₂ each independently is (C₁-C₁₆)alkyl, (C₂-C₁₆)alkenyl, or (C₂-C₁₆)alkynyl;
R₃ each independently is H, (C₁-C₈)alkyl, (C₃-C₁₀)cycloalkyl, 4-12-membered heterocyclyl, or (C₆-C₁₄)aryl, each of which other than H may optionally be substituted with —OH, —COR₄, —COOR₄, —OCOOR₄, —OCON(R₄)₂, —(C₁-C₈)alkylene-COOR₄, —CN, —NO₂, —SH, —SR₄, —(C₁-C₈)alkyl, —O—(C₁-C₈)alkyl, —N(R₄)₂, —CON(R₄)₂, —SO₂R₄, or —S(═O)R₄;
R₄ each independently is H, (C₁-C₈)alkyl, (C₃-C₁₀)cycloalkyl, 4-12-membered heterocyclyl, or (C₆-C₁₄)aryl; and
n and m each independently is an integer of 1 to 3.

22.-36. (canceled)

37. The method of claim 21, comprising administering a compound of the general formula I, wherein n is 1; R₂ each is methyl; R₁ linked to the carbon atom at position 3 of the pyrrolidine ring is the nitric oxide donor group —CH₂—ONO₂; and R₁ linked to the carbon atom at position 4 of the pyrrolidine ring is H, or an enantiomer, diastereomer, racemate, or pharmaceutically acceptable salt or solvate thereof.

38. The method of claim 11, for treatment of chlorine inhalational lung injury.

39.-41. (canceled)

42. The pharmaceutical composition of any claim 10, for intravenous, intramuscular, intraperitoneal, intrathecal, intrapleural, subcutaneous, intratracheal, or inhalational administration.

43.-44. (canceled)

45. The method of claim 21, wherein the method is applicable to treat chlorine inhalational lung injury.
